(19) United States
(12) Patent Application Publication
(10) Pub. No.: US 2012/0083557 A1
(43) Pub. Date: Apr. 5, 2012

(54) STERICALLY HINDERED AMINE STABILIZERS

(75) Inventor: Kai-Uwe Schoening, Oberwil (CH)

(73) Assignee: BASF SE, Ludwigshafen (DE)

(21) Appl. No.: 13/376,849

(22) PCT Filed: Jun. 1, 2010
(86) PCT No.: PCT/EP2010/057622

§ 371 Date: Dec. 8, 2011

(30) Foreign Application Priority Data
Jun. 8, 2009 (EP) 09162187.0

(51) Int. Cl. C08K 5/3435; C07D 401/14; C07D 491/22; C07D 491/113; C07D 498/10; C08L 23/06; C07D 498/04; C07D 487/16; C08K 5/3492; C08K 5/3467; C08K 5/357; C08L 23/12; C07D 401/12; C07D 403/14

(52) U.S. Cl. 524/89; 546/188; 546/16; 546/19; 544/198; 540/472; 544/83; 524/102; 524/94; 524/100; 524/97

(57) ABSTRACT

The instant invention pertains to hindered amine compounds having at least two nitrogen atoms with different basicity. One part is substituted on the N-atom by alkoxy moieties and the other part is substituted on the N-atom by a hydroxy-alkyl moiety. These materials are particularly effective in stabilizing polymers, especially thermoplastic polyolefins, against the deleterious effects of oxidative, thermal and actinic radiation. The compounds are also particularly effective in stabilizing acid catalyzed and ambient cured coatings systems.

The instant invention pertains to hindered amine compounds having at least two nitrogen atoms with different basicity. One part is substituted on the N-atom by alkoxy moieties and the other part is substituted on the N-atom by a hydroxy-alkyl moiety. These materials are particularly effective in stabilizing polymers, especially thermoplastic polyolefins, against the deleterious effects of oxidative, thermal and actinic radiation. The compounds are also particularly effective in stabilizing acid catalyzed and ambient cured coatings systems.

Conventional HALS (N—H) are excluded from certain applications due to their basic secondary ring nitrogen atom. Examples are acid catalyzed crosslinked polymers or ambient cured coatings.

The present invention solves this problem by providing novel NOR HALS compounds which combine the advantages of hydroxyl-alkyl-HALS and N-alkoxy HALS which have a significantly higher than state-of-the-art active nitrogen densitity and, in addition, a reduced basicity of the HALS ring nitrogen atoms.

The compounds prepared can be used as stabilizers for plastics, coatings and home and personal care applications.

The instant compounds, because of their low bascity, are of particular value in the stabilization of polyolefins and automotive coating compositions where the activity of the more basic hindered amine stabilizers is significantly reduced because of interaction with the polymer substrate or acid catalysts needed for curing such substrates.

Examples of polyolefin compositions in which the instant compounds are effective include flame retardant polyolefins where acidic residues from the decomposition of the halogenated flame retardants deactivate hindered amines not having the N—OR group, greenhouse films and agricultural mulch films where acidic residues from pesticides or sulfur dioxide treatment interfere with the activity of “normal” hindered amine stabilizers, and in thermoplastic polyolefins where pigment interactions with basic hindered amine stabilizers may occur. Examples of coating compositions in which the instant compounds are effective include melamine crosslinked thermoset acrylic resins, which are cured using strong acids that interact with basic hindered amine stabilizers. The instant compounds are also effective in acrylic alkyd or polyester resins with isocyanate crosslinking agents, and in epoxy resins with carboxylic acid, anhydride, or amine crosslinking agents.

Thus, current compounds are advantageously used in compositions also containing co-stabilizers, flame retardants (e.g. tris(3-bromo-2,2-bis(bromomethyl)propyl)phosphate, decabromodiphenyl oxide, ethylene bis-(tetrabromophthalimide), or ethylene bis-(dibromo-norbornanedicarboximide)), catalysts (e.g. acids like toluene sulfonic acid, metal driers or amines), fillers, fatty acid salts (e. g. calcium stearate)or in agricultural applications wherein pesticides and/or sulfur-containing acids and or metal ions (e. g. iron) come in contact with the stabilized polymer.

One aspect of the invention is a compound of formula (I)

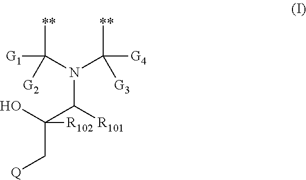

wherein
G₁, G₂, G₃ and G₄ are independently alkyl of 1 to 4 carbon atoms, or G₁ and G₂ and/or G₃ and G₄ together are pentamethylene;
R₁₀₁ and R₁₀₂ are independently hydrogen or C₁-C₈alkyl; and
Q is a group of formula (II), (III), (IV) or (V)

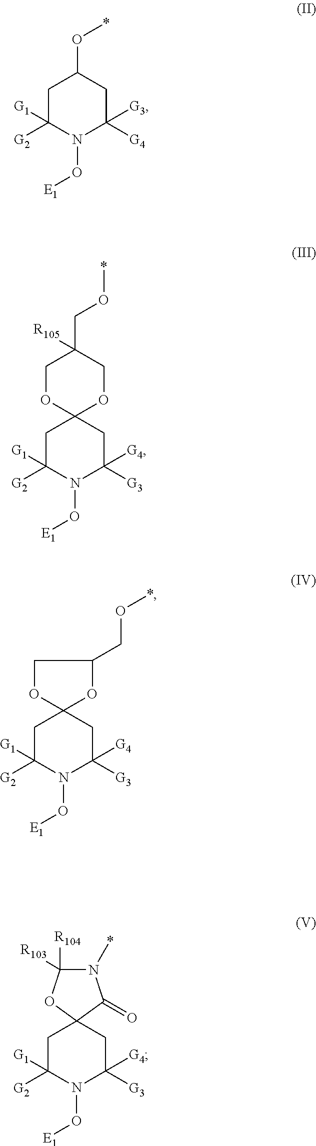

R₁₀₃ and R₁₀₄ are independently —C₃-C₁₂cycloalkyl or C₁-C₁₈alkyl, which is unsubstituted or substituted with halogen, nitro or —C(O)H;
R₁₀₅ is hydrogen, ethyl or methyl;
E₁ is straight or branched chain C₁-C₂₄alkyl, C₂-C₁₈alkenyl, C₂-C₁₈alkinyl, C₃-C₁₂cycloalkyl, C₅-C₁₂cycloalkenyl, phenyl, naphthyl or C₇-C₁₅phenylalkyl which each may be unsubstituted or substituted by 1 to 3 OH groups; or
E₁ is C₂-C₂₄alkyl which is substituted by NO₂, POR′ or NHR′ wherein R′ is C₁-C₁₂alkyl;
* indicates the point of attachment; and
** indicates the point of attachment to one or two organic residues containing in total 1-500 carbon atoms and optionally 1-200 heteroatoms.

For example the compound is of formula (I′)

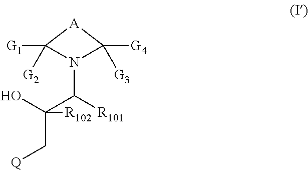

wherein A is a divalent group required to form a cyclic or heterocyclic 5-, 6- or 7-membered ring, which is unsubstituted or substituted by —OH, ═O or by one or two organic residues containing in total 1-500 carbon atoms and optionally 1-200 heteroatoms.

Preferred is a compound which is of formulae 1 to 16

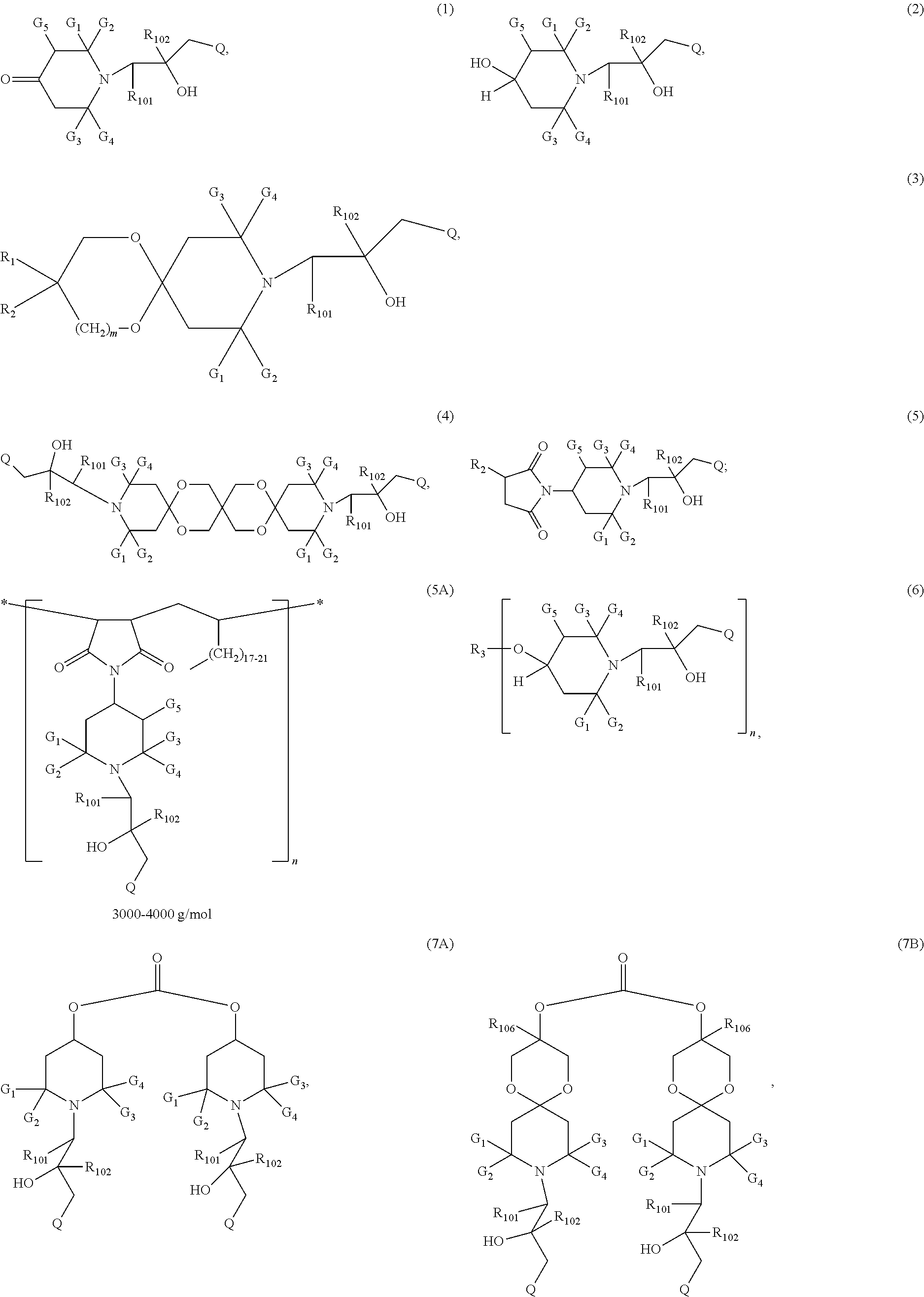

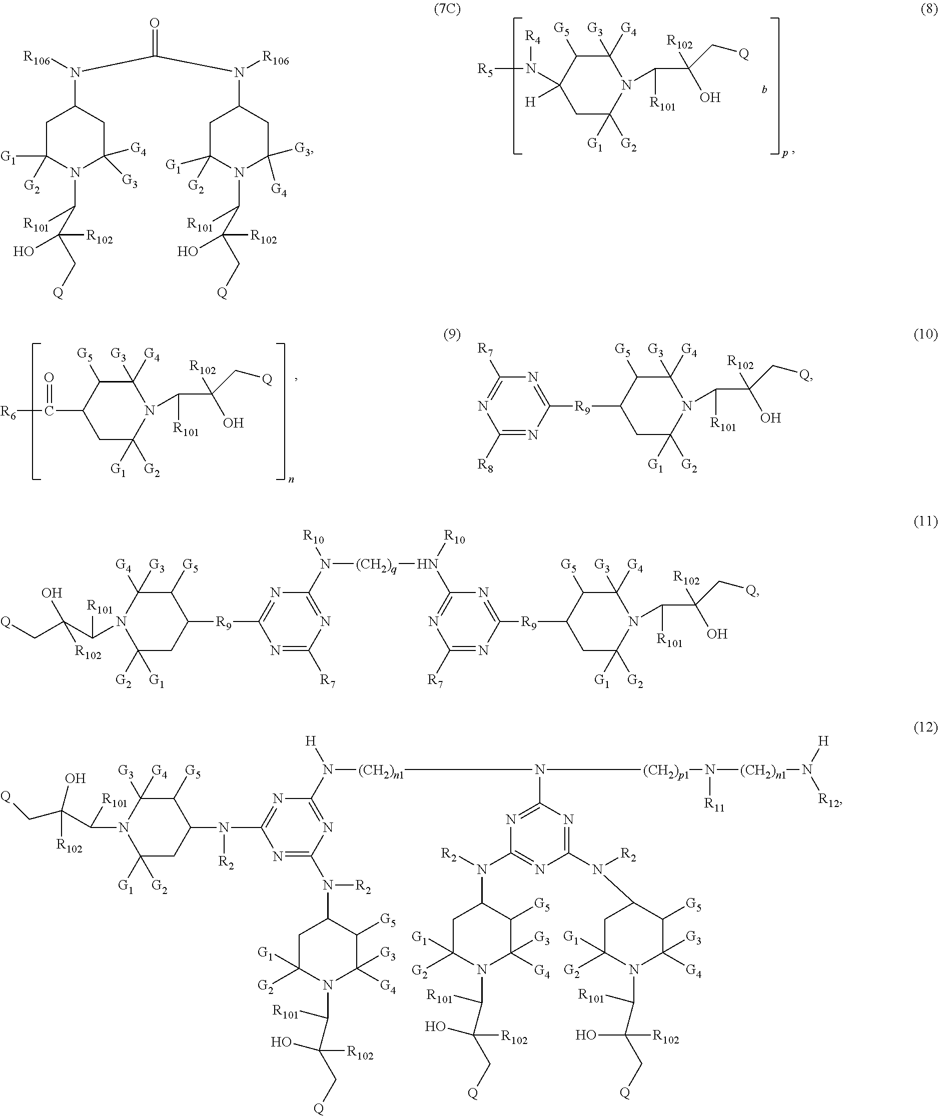

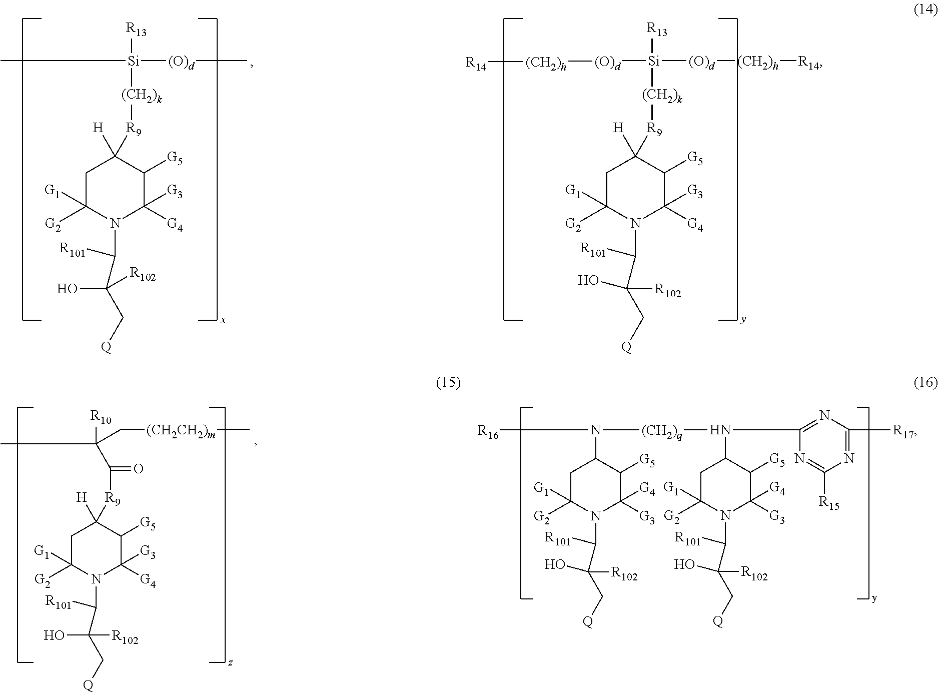

wherein
G₁, G₂, G₃, G₄, R₁₀₁, R₁₀₂, R₁₀₃, R₁₀₄, R₁₀₅ and Q are as defined in claim 1
G₅ is hydrogen or methyl;
R₁₀₆ is hydrogen, alkyl of 1 to 12 carbon atoms or alkenyl of 2 to 12 carbon atoms;

in each of the formulas

m is 0 or 1;

R₁ is hydrogen, hydroxyl or hydroxymethyl;

R₂ is hydrogen, alkyl of 1 to 12 carbon atoms or alkenyl of 2 to 12 carbon atoms;

n is 1 to 4;

when n is 1,

R₃ is alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;

when n is 2,

R₃ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;

when n is 3,

R₃ is a trivalent acyl radical acyl radical of an aliphatic, unsaturated aliphatic, or cycloaliphatic tricarboxylic acid or tricarbamic acid containing 6-18 carbon atoms, or a trivalent acyl radical of an aromatic tricarboxylic or tricarbamic acid containing 9-18 carbon atoms, or R₃ is a trivalent acyl radical of a tris(alkylcarbamic acid) derivative of cyanuric acid containing 12-24 carbon atoms, such as 1,3,5-tris[6-carboxyaminohexyl]-2,4,6-trioxo-s-triazine;

when n is 4,

R₃ is a tetravalent acyl radical of an aliphatic or unsaturated aliphatic tetracarboxylic acid, or R₃ is a tetravalent acyl radical of an aromatic tetracarboxylic acid containing 10 to 18 carbon atoms;

p is 1 to 3,

R₄ is hydrogen, alkyl of 1 to 18 carbon atoms or acyl of 2 to 6 carbon atoms;

when p is 1,

R₅ is hydrogen, alkyl of 1 to 18 carbon atoms, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, an acyl radical of an aromatic carboxylic acid containing 7 to 15 carbon atoms, or R₄ and R₅ together are —(CH₂)₅CO—, phthaloyl or a divalent acyl radical of maleic acid;

when p is 2,

R₅ is alkylene of 2 to 12 carbon atoms, a divalent acyl radical of an aliphatic or un- saturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;

when p is 3,

R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;

when n is 1,

R₆ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms,

when n is 2,

R₆ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)— of 2 to 18 carbon atoms, or R₆ is 4-methyl-1,3-phenylenediamino,

when n is 3,

R₆ is a trivalent alkoxy radical of a saturated or unsaturated aliphatic triol containing 3 to 18 carbon atoms,

when n is 4,

R₆ is a tetravalent alkoxy radical of a saturated or unsaturated aliphatic tetraol containing 4 to 18 carbon atoms,

R₇ and R₈ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms,

R₉ is a divalent oxygen atom, or R₉ is a divalent nitrogen atom substituted by either hydrogen, alkyl of 1 to 12 carbon atoms or T₁

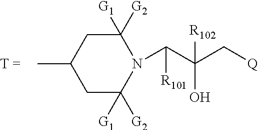

R₁₀ is hydrogen or methyl,

q is 2 to 8,

R₁₁ and R₁₂ are independently hydrogen or the group T₂

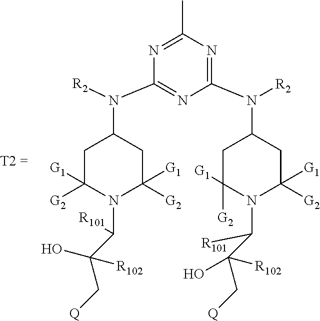

n1 is a number from 1 to 6;
p1 is a number from 1 to 6;

R₁₃ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;

R₁₄ is hydrogen or silyl substituted three times independently by hydrogen, by phenyl, by alkyl of 1 to 4 carbon atoms or by alkoxy of 1 to 4 carbon atoms;

d is 0 or 1;

h is 0 to 4;

k is 0 to 5;

x is 3 to 6;

y is 1 to 10;

z is an integer such that the compound has a molecular weight of 1000 to 4000 amu,

R₁₅ is morpholino, piperidino, 1-piperizinyl, alkylamino of 1 to 8 carbon atoms, especially branched alkylamino of 3 to 8 carbon atoms such as tert-octylamino, —N(alkyl)T₁ with alkyl of 1 to 8 carbon atoms substituted by T₁ or —N(alkyl)₂ of 2 to 16 carbon atoms,

R₁₆ is hydrogen, acyl of 2 to 4 carbon atoms, carbamoyl substituted by alkyl of 1 to 4 carbon atoms, s-triazinyl substituted once by chlorine and once by R₁₅, or s-triazinyl substituted twice by R₁₅ with the condition that the two R₁₅ substituents may be different;

R₁₇ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃

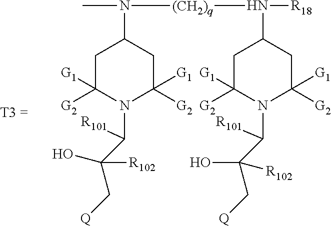

q is 2 to 8; and

R₁₈ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms.

Preferably R₂ is hydrogen, alkyl of 1 to 12 carbon atoms.

For example R₁₀₁ and R₁₀₂ are hydrogen.

For instance R₁₀₃ and R₁₀₄ are C₁-C₁₈alkyl.

Preferred is a compound wherein G₁ and G₃ are ethyl and G₂, G₄ and G₅ are methyl or G₁ and G₂ are methyl, G₃ and G₄ are ethyl and G₅ is hydrogen or G₁, G₂, G₃ and G₄ are methyl and G₅ is hydrogen.

In a specific embodiment G₁, G₂, G₃, G₄ are methyl and G₅ is hydrogen.

Preference is given to a compound wherein G₁, G₂, G₃ and G₄ are methyl, R₁₀₁ and R₁₀₂ are hydrogen and R₁₀₃ and R₁₀₄ are independently C₁-C₁₈alkyl.

Preferably in formula (3), m is 0, R₁ is hydrogen or hydroxymethyl, and R₂ is hydrogen; or m is 1, R₁ is hydroxy or hydroxymethyl, and R₂ is hydrogen, methyl or ethyl.

Preferably in formula (5), R₂ is hydrogen or dodecyl.

Preferably in formula (6), n is 1-3, and when n is 1, R₃ is allyl, glycidyl, acryloyl, methacryloyl, octadecanoyl, hexadecanoyl, tetradecanoyl, dodecanoyl, methoxycarbonylpropionyl, methoxycarbonylbutyryl, methoxycarbonylpentanoyl or methoxycarbonylnonanoyl; or when n is 2, R₃ is succinyl, glutaryl, adipoyl, sebacoyl, 1,6-hexanedicarbamoyl, cis- or trans-5-carbamoyl-1-(carbamoylmethyl)-1,3,3-trimethylcyclohexane or toluene-2,4-dicarbamoyl; or when n is 3, R₃ is 1,3,5-tris(6-carbamoylhexyl)-2,4,6-trioxo-s-triazine.

Most preferably in formula (6), when n is 1, R₃ is acryloyl, methacrloyl, glycidyl, octadecanoyl, hexadecanoyl, methoxycarbonylpropionyl, methoxycarbonylbutyryl, methoxycarbonyl-pentanoyl or methoxycarbonylnonanoyl; or when n is 2, R₃ is succinyl, glutaryl, adipoyl, sebacoyl, 1,6-hexanedicarbamoyl or cis- or trans-5-carbamoyl-1-(carbamoylmethyl)-1,3,3-trimethylcyclohexane or toluene-2,4-dicarbamoyl; or when n is 3, R₃ is 1,3,5-tris(6-carbamoylhexyl)-2,4,6-trioxo-s-triazine.

Preferably in formula (8), p is 1 or 2, and when p is 1, R₄ is hydrogen and R₅ is butyl; or R₄ and R₅ together are the divalent acyl radical of maleic acid; or when p is 2, R₄ is hydrogen or acetyl, and R₅ is 1,6-hexanediyl.

Most preferably in formula (8), p is 1 or 2, and when p is 1, R₄ is hydrogen and R₅ is hydrogen or butyl; or when p is 2, R₄ is hydrogen, and R₅ is 1,6-hexanediyl.

Preferably in formula (9), n is 1 or 2, and when n is 1, R₆ is ethoxy, 6-methyl-1-heptyloxy, ethylamino, butylamino or octylamino; or when n is 2, R₆ is 1,2-ethanedioxy, 1,4-butanedioxy, ethylenediamino, hexamethylenediamino, or 4-methyl-1,3-phenylenediamino.

Preferably in formula (10), R₇ and R₈ are independently chlorine, octylamino, tert-octylamino or amino substituted by T₁ and ethyl, butyl or dodecyl; and R₉ is a divalent nitrogen atom substituted by ethyl, butyl or dodecyl.

Preferably in formula (11), q is 2, 4 or 6, R₇ is chlorine, octylamino, octadecylamino or amino substituted by T₁ and ethyl, butyl or dodecyl; and R₁₀ is hydrogen.

Preferably in formula (12), n1 is 3, p1 is 2, R₂ is ethyl, butyl or dodecyl; and one of R₁₁ or R₁₂ is T₂, and the other is hydrogen.

Preferably in formula (13), k is 3, R₉ is a divalent oxygen atom or is a divalent nitrogen atom substituted by ethyl, butyl or dodecyl, R₁₃ is hydrogen or methyl, and when d is 0, x is 5 or 6, and when d is 1, x is 3 or 4.

Preferably in formula (14), d is 0 or 1, h is 0-2, k is 0 or 3, y is 1-8, R₉ is a divalent oxygen atom or a divalent nitrogen atom substituted by ethyl, butyl or dodecyl, R₁₃ is hydrogen, methyl, ethyl, methoxy or ethoxy, and R₁₄ is hydrogen or trimethylsilyl.

Preferably in formula (15), R₉ is a divalent oxygen atom, R₁₀ is hydrogen or methyl, m is 0 and z is an integer such that the molecular weight of the compound is 1500-3000 amu.

Preferably in formula (16) q is 6, y is 1-7, R₁₅ is tert-octylamino, morpholino, amino substituted by T₁ and butyl, which may also be designated as T₁-butylamino, R₁₆ is hydrogen, acetyl, ethylcarbamoyl, 2,4-bis(dibutylamino)-s-triazinyl, 2,4-bis(diethylamino)-s-triazinyl, s-triazinyl substituted twice by T₁-butylamino or s-triazinyl substituted once by diethylamino or dibutylamino and once by T₁-butylamino, R₁₇ is dibutylamino, diethylamino, T₁-butylamino or R₁₇ is T₃ where R₁₈ is acetyl or ethylcarbamoyl.

Most preferably in formula (8), p is 1 or 2, and when p is 1, R₄ is hydrogen and R₅ is hydrogen or butyl; or when p is 2, R₄ is hydrogen, and R₅ is 1,6-hexanediyl.

Most preferably in formula (10), R₇ is chlorine, octylamino or T₁-butylamino, R₈ is chlorine or T₁-butylamino, and R₉ is a divalent nitrogen atom substituted by butyl.

Most preferably in formula (11), q is 6, R₇ is T₁-butylamino; and R₁₀ is hydrogen.

Most preferably in formula (12), n1 is 3, p1 is 2, and one of R₁₁ or R₁₂ is T₂, and the other is hydrogen.

Most preferably in formula (13), k is 3, R₉ is a divalent oxygen atom, R₁₃ is hydrogen or methyl, and d is 0, x is 5 or 6, and when d is 1, x is 3 or 4.

Most preferably in formula (14), d is 0 or 1, h is 0-2, k is 0 or 3, y is 1-8, R₉ is a divalent oxygen atom, R₁₃ is hydrogen, methyl, ethyl, methoxy or ethoxy, and R₁₄ is hydrogen or trimethylsilyl.

Most preferably in formula (16) q is 6, y is 1-7, R₁₅ is T₁-butylamino, R₁₆ is hydrogen, acetyl, ethylcarbamoyl, 2,4-bis(dibutylamino)-s-triazinyl, 2,4-bis(diethylamino)-s-triazinyl, s-triazinyl substituted twice by T₁-butylamino or s-triazinyl substituted once by diethylamino or dibutylamino and once by T₁-butylamino, R₁₇ is dibutylamino, diethylamino, T₁-butylamino or R₁₇ is T₃ where R₁₈ is acetyl or ethylcarbamoyl.

Especially preferred compounds of formula (6) are those where n is 1, R₃ is acryloyl, methacryloyl, glycidyl, octadecanoyl, hexadecanoyl, methoxycarbonylpropionyl or methoxycarbonylbutyryl or methoxycarbonylpentanoyl, and where n is 2, R₃ is succinyl, glutaryl, adipoyl or sebacoyl.

Especially preferred compounds of formula (8) are those where R₄ is hydrogen, and when p is 1, R₅ is hydrogen or butyl, or when p is 2, R₅ is hexamethylene.

Especially preferred compounds of formula (10) are those where R₇ is chlorine, octylamino or T₁-butylamino, R₈ is T₁-butylamino, and R₉ is a divalent nitrogen atom substituted by butyl.

Especially preferred compounds of formula (11) are those where q is 6, R₇ is T₁-butylamino and R₁₀ is hydrogen.

Especially preferred compounds of formula (12) are those where n1 is 3, p1 is 2, one of R₁₁ or R₁₂ is T₂ and the other is hydrogen.

Especially preferred compounds of formula (13) are those where d is 1, k is 3, x is 3 or 4, R₉ is divalent oxygen atom, and R₁₃ is methyl.

Especially preferred compounds of formula (14) are those where k is 3, y is 4-8. R₉ is a divalent oxygen atom, R₁₃ is hydrogen or methyl, d and h are 0, R₁₄ is hydrogen, or d is 1 and h is 0, and R₁₄ is trimethylsilyl.

Especially preferred compounds of formula (15) are those where m is 0, R₉ is a divalent oxygen atom, R₁₀ is hydrogen or methyl, and z is an integer such that the molecular weight of the compound is 1500-3000 g/mol.

Especially preferred compounds of formula (16) are those where q is 6, y is 1-7, R₁₅ is T₁-butylamino, R₁₆ is hydrogen, acetyl, ethylcarbamoyl, 2,4-bis(dibutylamino)-s-triazinyl, 2,4-bis(diethylamino)-s-triazinyl, s-triazinyl substituted twice by T₁-butylamino or s-triazinyl substituted once by diethylamino or dibutylamino and once by T₁-butylamino, R₁₇ is dibutyl-amino, diethylamino, or T₃ where R₁₈ is acetyl or ethylcarbamoyl.

Preferably Q is a group of formula (II) or (III).

Preferably E₁ is straight or branched C₁-C₁₈alkyl.

Preferred are the compounds of formula (1), (2), (3), (6), (7A), (7B), (10) (12) or (16).

Individual compounds are given below

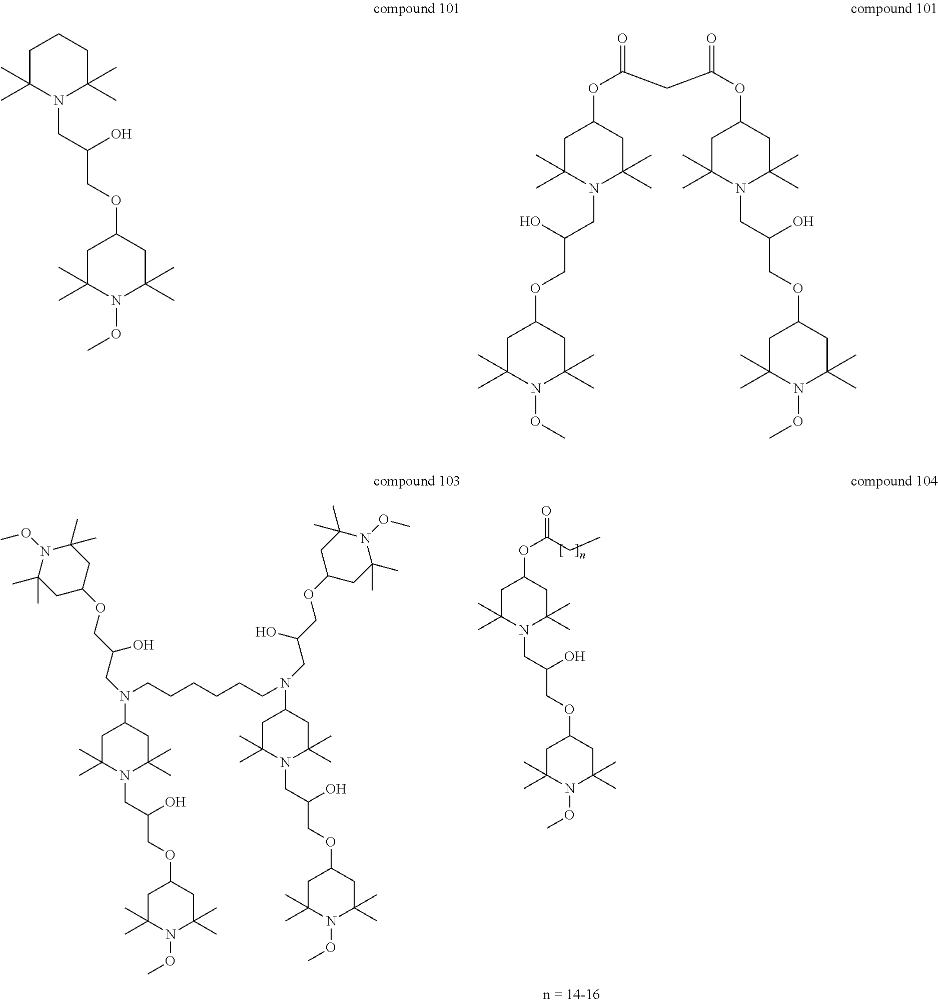

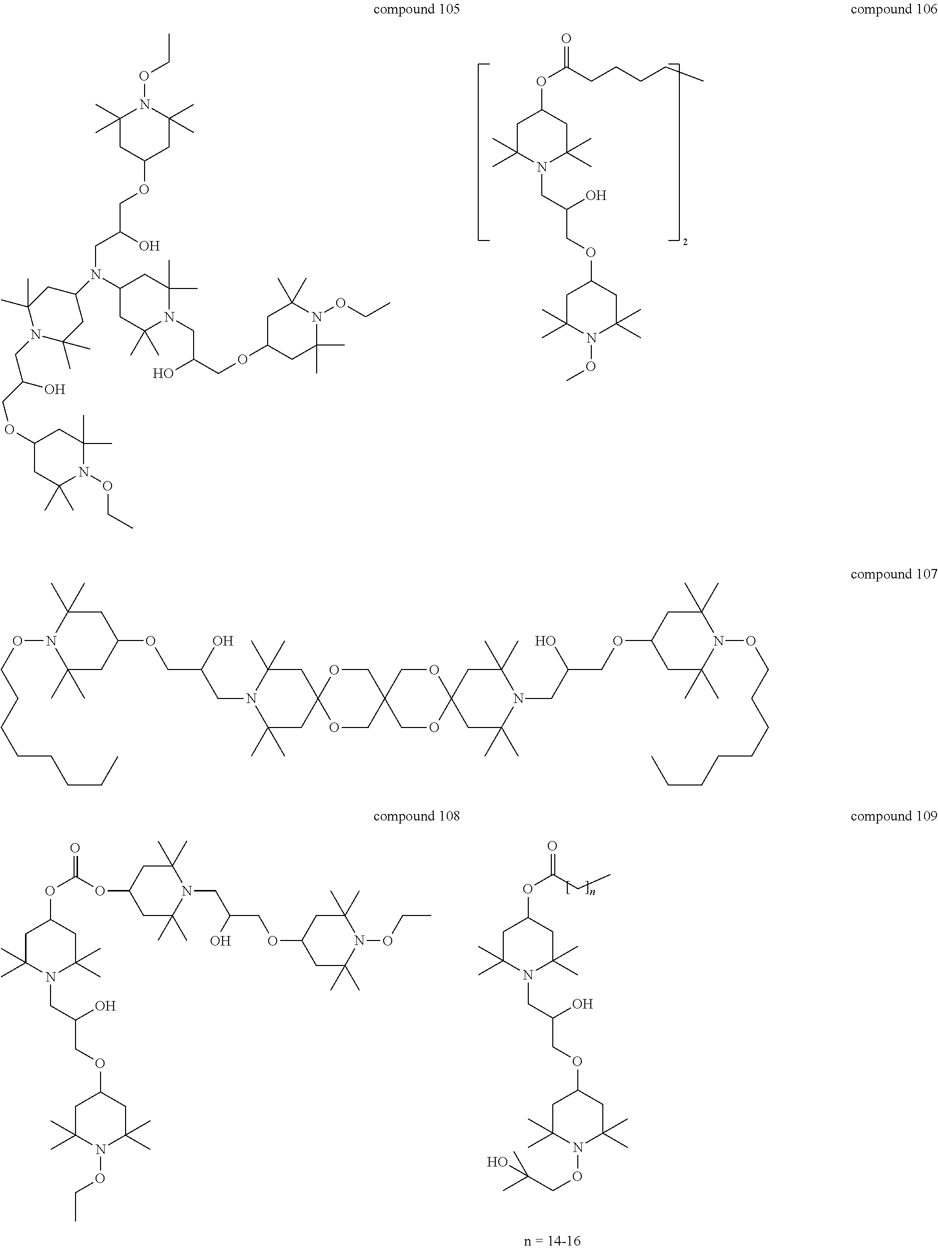

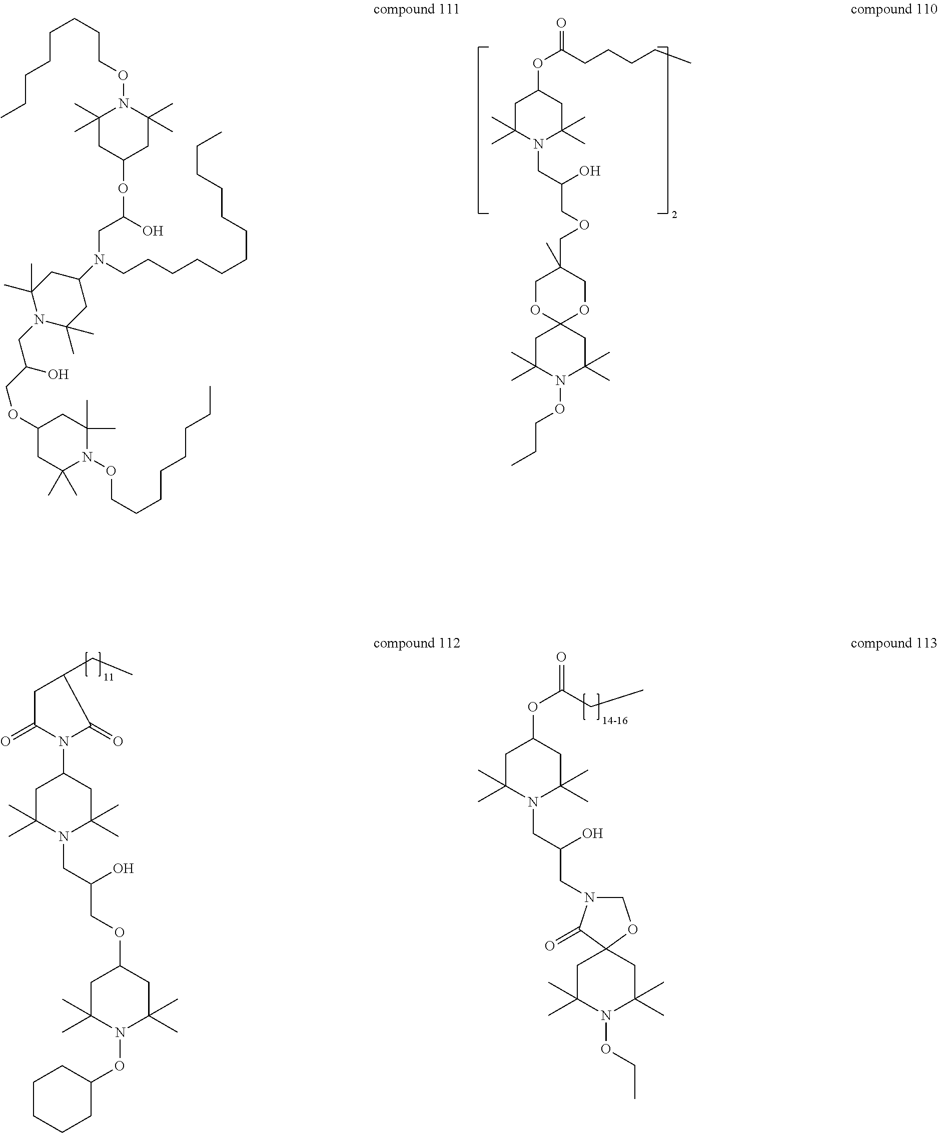

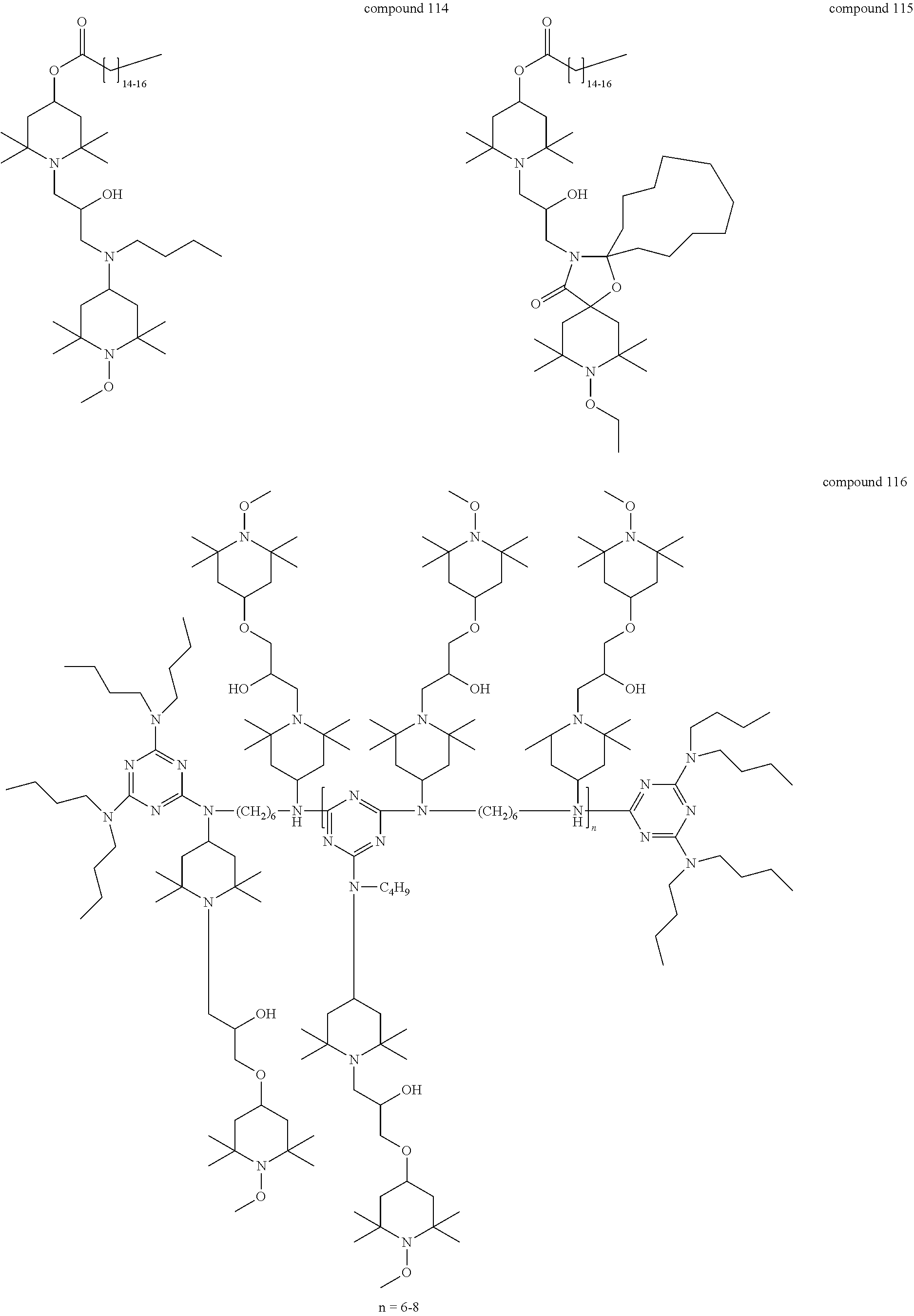

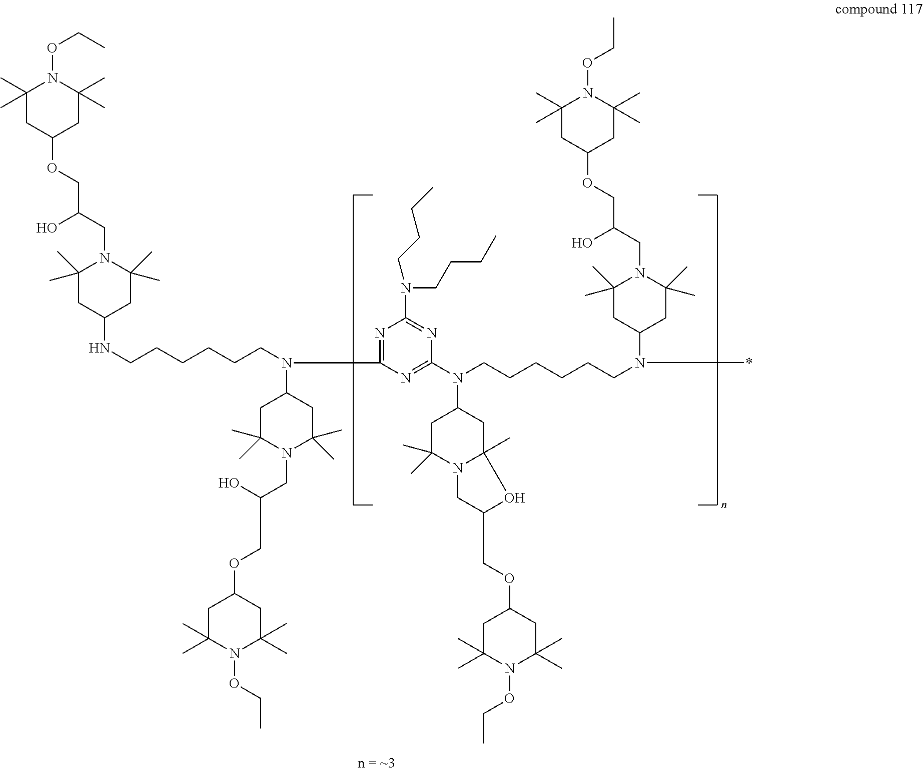

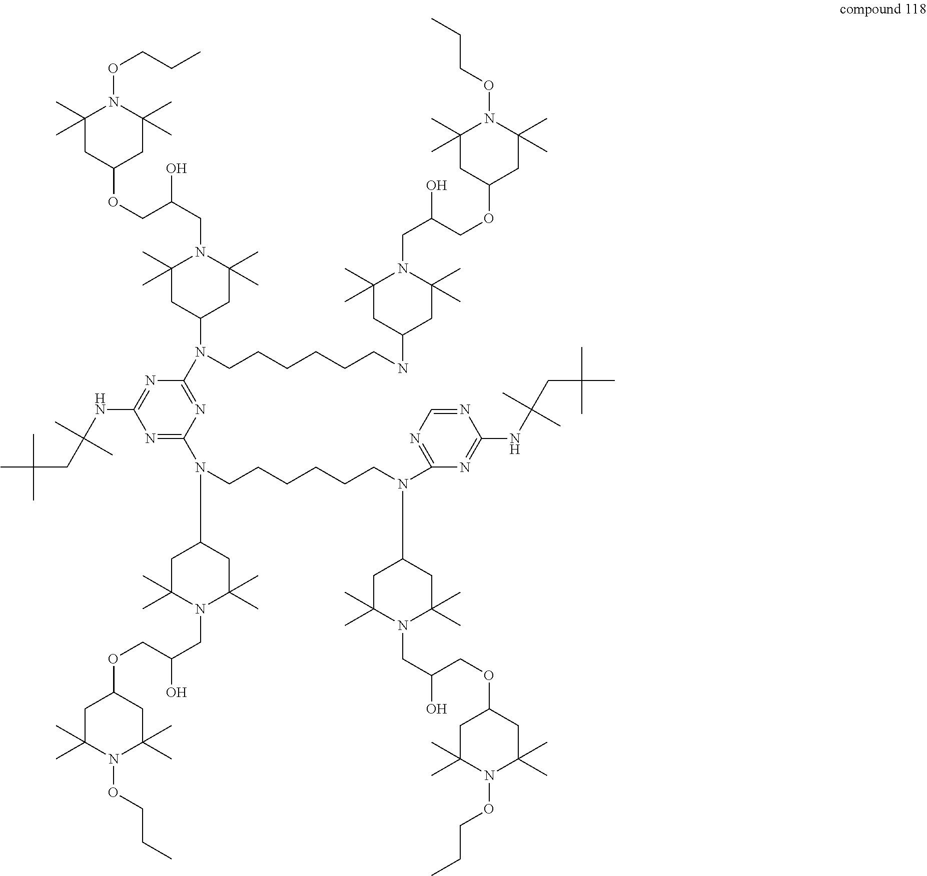

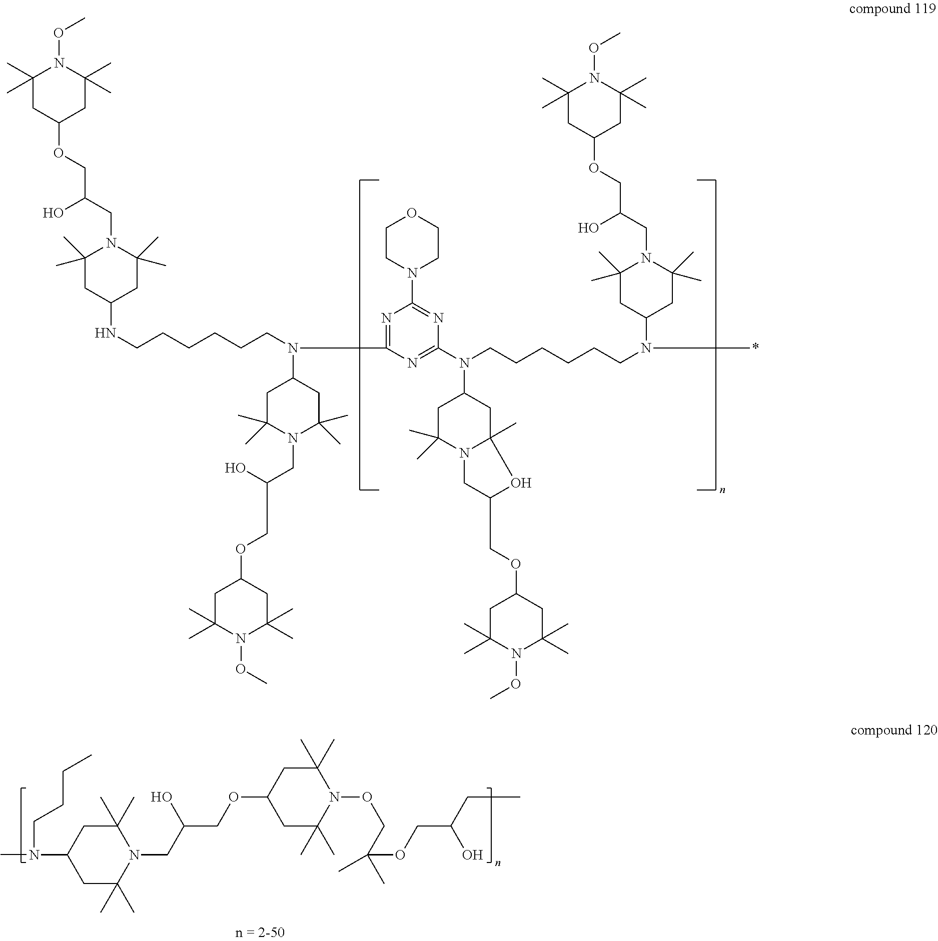

The compounds mentioned above can principally be prepared by reacting a sterically hindered secondary amine compound with a compound of formula (VII)

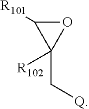

Therefore, a further aspect of the invention is a process for the preparation of a compound of formula (I)

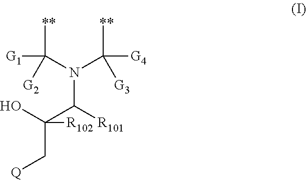

comprising reacting a sterically hindered amine containing the structural element of formula (VI)

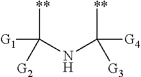

wherein

G₁, G₂, G₃ and G₄ are independently alkyl of 1 to 4 carbon atoms, or G₁and G₂ and/or G₃ and G₄ together are pentamethylene; and

** indicates the point of attachment to one or two organic residues containing in total 1-500 carbon atoms and optionally 1-200 heteroatoms;
with a compound of formula (VII)

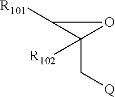

wherein

R₁₀₁ and R₁₀₂ are independently hydrogen or C₁-C₈alkyl; and

Q is a group of formula (II), (III), (IV) or (V)

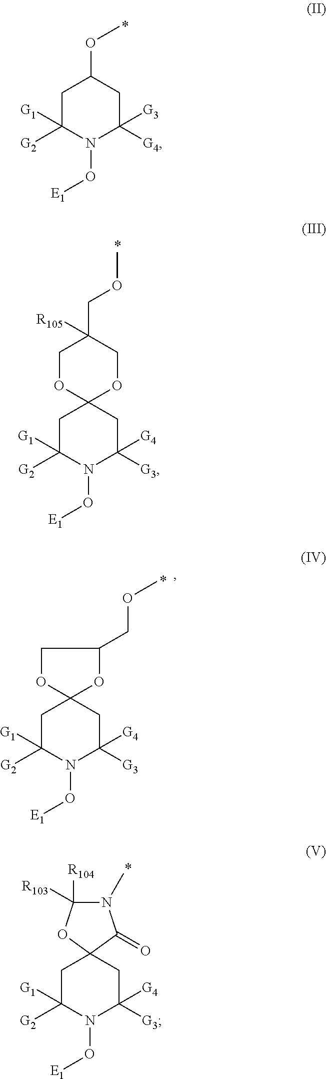

R₁₀₃ and R₁₀₄ are independently —C₃-C₇cycloalkyl or C₁-C₁₈alkyl, which is unsubstituted or substituted with halogen, nitro or —C(O)H;

R₁₀₅ is ethyl or methyl;

E₁ is straight or branched chain C₁-C₂₄alkyl, C₂-C₁₈alkenyl, C₂-C₁₈alkinyl, C₅-C₁₂cycloalkyl, C₅-C₁₂cycloalkenyl, phenyl, naphthyl or C₇-C₁₅phenylalkyl which each may be unsubstituted or substituted by 1 to 3 OH groups; and

* indicates the point of attachment;
in the presence of a Lewis acid as catalyst.

The reaction can be carried out with any sterically hindered amine compound. Preferred are the compounds of formulae 1-16 outlined above wherein the substitution at the nitrogen atom is hydrogen. Suitable starting materials are, for example, the compounds of formula (6′)

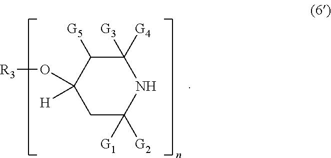

The starting materials for the other compounds can be deduced in analogy. Their preparation is essentially known and they are partially commercially available. All of them can be prepared by known processes. Their preparation is disclosed, for example, in:

U.S. Pat. No. 5,679,733, U.S. Pat. No. 3,640,928, U.S. Pat. No. 4,198,334, U.S. Pat. No. 5,204,473, U.S. Pat. No. 4,619,958, U.S. Pat. No. 4,110,306, U.S. Pat. No. 4,110,334, U.S. Pat. No. 4,689,416, U.S. Pat. No. 4,408,051, SU-A-768,175 (Derwent 88-138,751/20), U.S. Pat. No. 5,049,604, U.S. Pat. No. 4,769,457, U.S. Pat. No. 4,356,307, U.S. Pat. No. 4,619,956, U.S. Pat. No. 5,182,390, GB-A-2,269,819, U.S. Pat. No. 4,292,240, U.S. Pat. No. 5,026,849, U.S. Pat. No. 5,071,981, U.S. Pat. No. 4,547,538, U.S. Pat. No. 4,976,889, U.S. Pat. No. 4,086,204, U.S. Pat. No. 6,046,304, U.S. Pat. No. 4,331,586, U.S. Pat. No. 4,108,829, U.S. Pat. No. 5,051,458, WO-A-94/12,544 (Derwent 94-177,274/22), DD-A-262,439 (Derwent 89-122,983/17), U.S. Pat. No. 4,857,595, U.S. Pat. No. 4,529,760 , U.S. Pat. No. 4,477,615 , CAS 136,504-96-6, U.S. Pat. No. 4,233,412, U.S. Pat. No. 4,340,534, WO-A-98/51,690 and EP-A-1,803, in particular U.S. Pat. No. 4,442,250 or U.S. Pat. No. 6,046,304.

The sterically hindered secondary amine compounds are, for example, selected from the group consisting of the following commercial products:

DASTIB 845 (RTM), TINUVIN 770 (RTM), CHIMASSORB 944 (RTM), CHIMASSORB 2020 (RTM), MARK LA 57 (RTM), MARK LA 77 (RTM), MARK LA 67 (RTM), MARK LA 87 (RTM), HOSTAVIN 3055 (RTM), HOSTAVIN 3050 (RTM), HOSTAVIN 3052 (RTM), UVINUL 4050 (RTM), UVINUL 5050 (RTM), CYASORB UV 3853 (RTM), CYASORB UV 3346 (RTM).

The compounds of formula (VII)

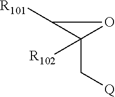

are prepared from alkoxyamines the preparation of which is described in WO 2008/003602. These alkoxyamines having a functional group such as OH or NH are reacted with, for example, epichlorhydrin to form compounds of formula (VII).

The preparation of the compounds of formula (I) is, for example, carried out at a temperature of from 20° C. to 280° C., preferably of from 80° C. to 180° C. and especially of from 120° C. to 150° C., preferably 80-180° C. and most preferably 120-150° C.

Preferably, atmospheric pressure is applied. However, a pressure from 1 to 100 bar can be applied, for example 1 to 20 bar.

The reaction time is typically between 1 to 48 hours.

The molar ratio between sterically hindered secondary amine (compound of formula VI) and epoxide (compound of formula VII) is, for example, from 1:1-1:10, preferably from 1:1-1:2, most preferably 1:1.

The reaction is typically carried out in a solvent. Suitable solvents are polar and apolar solvents, aromatic and aliphatic solvents. Examples are H₂O, tert.-butanol, 2-propanol, toluene, hexane, acetone, MTBE, dioxan, dimethylformamide, polyethyleneglycol. Preferred are apolar solvents with high boiling point, such as xylene or toluene (bp>100° C.). Most preferred are xylene, chlorobenzene, toluene (under pressure), high boiling alkanes, tetraline.

In some cases the reaction may also be carried out without solvent, in particular when the starting materials are liquid or low melting solids.

In some cases oligomeric or polymeric structures can be obtained in varying amounts in the epoxide ring opening process. This is the case when the initially formed alkoxide reacts with further epoxides instead of being transformed into a hydroxy group. Instances where this may happen are reactions were high concentrations of starting materials are employed (e.g. reactions without solvent). Minor amounts of these oligomeric or polymeric compounds are always present in the final product, but represent no limitation of the activity or the intended use.

The starting materials may be added simultaneously together with the catalyst or the epoxide and/or catalyst may be added over the course of time.

The reaction is carried out in the presence of a catalyst. As catalyst all lewis acids that promote epoxide ring opening come into consideration. Examples are ZnCl₂, AIEt₃, LaCl₃, La(OTf)₃, SC(OTf)₃, Ti(alkoxy)₄, bases (tert. Amines, Et₃N), LiCl, Ca(OTf)₂, Mg(OTf)₂, Al(CF₃SO₃)₃, Co-Salen, Ammonium decatungstocerate (IV), solid catalyst (W-200-N alumina, basic alumina, rare earth metal oxides, heteropoly acids, poloxometallates, hydrotalcites, zeolites, silica), K₂CO₃, CsF on Celite; acids, such as HCl, cyanuric chloride. Most preferred is ZnCl₂

The amount of catalyst used is typically 0.001-10 weight % relative to the weight of the sterically hindered secondary amine (compound of formula VI), preferably 0.1-3.5 weight % Most preferably 0.5-2 weight %.

Such ring opening reactions of epoxides are principally known and, for example, described by: Laguerre, M.; Boyer, C.; Carpy, A.; Leger, J. M.; Panconi, E.; et al.; EJMCA5; European Journal of Medicinal Chemistry; English; 28; 1; 1993; 81 - 85; Corral, C.; El-Ashmawy, M. B.; Lissavetzky, J.; Bravo, L.; Darias, V.; Martin, D.; FRPSAX; Farmaco, Edizione Scientifica; English; 42; 4; 1987; 267-276 and

Lohmann, D.; Lehmann, D.; Morgenstern, E.; Faust, G.; PHARAT; Pharmazie; German; 45; 1990; 401-403.

A further aspect of the invention is a composition which comprises

(a) an organic polymer subject to the adverse effects of heat, oxygen and light, and

(b) one or more compounds according to formula (I) of claim 1, of formula (I′) of claim 2 or of formulae 1 to 16 of claim 3.

Examples for organic polymers are given below.

1. Polymers of monoolefins and diolefins, for example polypropylene, polyisobutylene, polybut-1-ene, poly-4-methylpent-1-ene, polyvinylcyclohexane, polyisoprene or polybutadiene, as well as polymers of cycloolefins, for instance of cyclopentene or norbornene, polyethylene (which optionally can be crosslinked), for example high density polyethylene (HDPE), high density and high molecular weight polyethylene (HDPE-HMW), high density and ultrahigh molecular weight polyethylene (HDPE-UHMW), medium density polyethylene (MDPE), low density polyethylene (LDPE), linear low density polyethylene (LLDPE), (VLDPE) and (ULDPE).

Polyolefins, i.e. the polymers of monoolefins exemplified in the preceding paragraph, preferably polyethylene and polypropylene, can be prepared by different, and especially by the following, methods:
a) radical polymerisation (normally under high pressure and at elevated temperature).
b) catalytic polymerisation using a catalyst that normally contains one or more than one metal of groups IVb, Vb, VIb or VIII of the Periodic Table. These metals usually have one or more than one ligand, typically oxides, halides, alcoholates, esters, ethers, amines, alkyls, alkenyls and/or aryls that may be either π- or σ-coordinated. These metal complexes may be in the free form or fixed on substrates, typically on activated magnesium chloride, titanium(III) chloride, alumina or silicon oxide. These catalysts may be soluble or insoluble in the polymerisation medium. The catalysts can be used by themselves in the polymerisation or further activators may be used, typically metal alkyls, metal hydrides, metal alkyl halides, metal alkyl oxides or metal alkyloxanes, said metals being elements of groups Ia, IIa and/or IIIa of the Periodic Table. The activators may be modified conveniently with further ester, ether, amine or silyl ether groups. These catalyst systems are usually termed Phillips, Standard Oil Indiana, Ziegler (-Natta), TNZ (DuPont), metallocene or single site catalysts (SSC).

2. Mixtures of the polymers mentioned under 1), for example mixtures of polypropylene with polyisobutylene, polypropylene with polyethylene (for example PP/HDPE, PP/LDPE) and mixtures of different types of polyethylene (for example LDPE/HDPE).

3. Copolymers of monoolefins and diolefins with each other or with other vinyl monomers, for example ethylene/propylene copolymers, linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, ethylene/vinylcyclohexane copolymers, ethylene/cycloolefin copolymers (e.g. ethylene/norbornene like COC), ethylene/1-olefins copolymers, where the 1-olefin is generated in-situ; propylene/butadiene copolymers, isobutylene/isoprene copolymers, ethylene/vinylcyclohexene copolymers, ethylene/alkyl acrylate copolymers, ethylene/alkyl methacrylate copolymers, ethylene/vinyl acetate copolymers or ethylene/acrylic acid copolymers and their salts (ionomers) as well as terpolymers of ethylene with propylene and a diene such as hexadiene, dicyclopentadiene or ethylidene-norbornene; and mixtures of such copolymers with one another and with polymers mentioned in 1) above, for example polypropylene/ethylene-propylene copolymers, LDPE/ethylene-vinyl acetate copolymers (EVA), LDPE/ethylene-acrylic acid copolymers (EAA), LLDPE/EVA, LLDPE/EAA and alternating or random polyalkylene/carbon monoxide copolymers and mixtures thereof with other polymers, for example polyamides.

4. Hydrocarbon resins (for example C₅-C₉) including hydrogenated modifications thereof (e.g. tackifiers) and mixtures of polyalkylenes and starch.

Homopolymers and copolymers from 1.)-4.) may have any stereostructure including syndio-tactic, isotactic, hemi-isotactic or atactic; where atactic polymers are preferred. Stereoblock polymers are also included.

5. Polystyrene, poly(p-methylstyrene), poly(α-methylstyrene).

6. Aromatic homopolymers and copolymers derived from vinyl aromatic monomers including styrene, α-methylstyrene, all isomers of vinyl toluene, especially p-vinyltoluene, all isomers of ethyl styrene, propyl styrene, vinyl biphenyl, vinyl naphthalene, and vinyl anthracene, and mixtures thereof. Homopolymers and copolymers may have any stereostructure including syndiotactic, isotactic, hemi-isotactic or atactic; where atactic polymers are preferred. Stereoblock polymers are also included.

6a. Copolymers including aforementioned vinyl aromatic monomers and comonomers selected from ethylene, propylene, dienes, nitriles, acids, maleic anhydrides, maleimides, vinyl acetate and vinyl chloride or acrylic derivatives and mixtures thereof, for example styrene/butadiene, styrene/acrylonitrile, styrene/ethylene (interpolymers), styrene/alkyl methacrylate, styrene/butadiene/alkyl acrylate, styrene/butadiene/alkyl methacrylate, styrene/maleic anhydride, styrene/acrylonitrile/methyl acrylate; mixtures of high impact strength of styrene copolymers and another polymer, for example a polyacrylate, a diene polymer or an ethylene/propylene/diene terpolymer; and block copolymers of styrene such as styrene/butadiene/styrene, styrene/isoprene/styrene, styrene/ethylene/butylene/styrene or styrene/ethylene/propylene/styrene.

6b. Hydrogenated aromatic polymers derived from hydrogenation of polymers mentioned under 6.), especially including polycyclohexylethylene (PCHE) prepared by hydrogenating atactic polystyrene, often referred to as polyvinylcyclohexane (PVCH).

6c. Hydrogenated aromatic polymers derived from hydrogenation of polymers mentioned under 6a.).

Homopolymers and copolymers may have any stereostructure including syndiotactic, isotactic, hemi-isotactic or atactic; where atactic polymers are preferred. Stereoblock polymers are also included.

7. Graft copolymers of vinyl aromatic monomers such as styrene or α-methylstyrene, for example styrene on polybutadiene, styrene on polybutadiene-styrene or polybutadiene-acrylonitrile copolymers; styrene and acrylonitrile (or methacrylonitrile) on polybutadiene; styrene, acrylonitrile and methyl methacrylate on polybutadiene; styrene and maleic anhydride on polybutadiene; styrene, acrylonitrile and maleic anhydride or maleimide on polybutadiene; styrene and maleimide on polybutadiene; styrene and alkyl acrylates or methacrylates on polybutadiene; styrene and acrylonitrile on ethylene/propylene/diene terpolymers; styrene and acrylonitrile on polyalkyl acrylates or polyalkyl methacrylates, styrene and acrylonitrile on acrylate/butadiene copolymers, as well as mixtures thereof with the copolymers listed under 6), for example the copolymer mixtures known as ABS, MBS, ASA or AES polymers.

8. Halogen-containing polymers such as polychloroprene, chlorinated rubbers, chlorinated and brominated copolymer of isobutylene-isoprene (halobutyl rubber), chlorinated or sulfo-chlorinated polyethylene, copolymers of ethylene and chlorinated ethylene, epichlorohydrin homo- and copolymers, especially polymers of halogen-containing vinyl compounds, for example polyvinyl chloride, polyvinylidene chloride, polyvinyl fluoride, polyvinylidene fluoride, as well as copolymers thereof such as vinyl chloride/vinylidene chloride, vinyl chloride/vinyl acetate or vinylidene chloride/vinyl acetate copolymers.

9. Polymers derived from α,β-unsaturated acids and derivatives thereof such as polyacrylates and polymethacrylates; polymethyl methacrylates, polyacrylamides and polyacrylonitriles, impact-modified with butyl acrylate.

10. Copolymers of the monomers mentioned under 9) with each other or with other unsaturated monomers, for example acrylonitrile/ butadiene copolymers, acrylonitrile/alkyl acrylate copolymers, acrylonitrile/alkoxyalkyl acrylate or acrylonitrile/vinyl halide copolymers or acrylonitrile/alkyl methacrylate/butadiene terpolymers.

11. Polymers derived from unsaturated alcohols and amines or the acyl derivatives or acetals thereof, for example polyvinyl alcohol, polyvinyl acetate, polyvinyl stearate, polyvinyl benzoate, polyvinyl maleate, polyvinyl butyral, polyallyl phthalate or polyallyl melamine; as well as their copolymers with olefins mentioned in 1) above.

12. Homopolymers and copolymers of cyclic ethers such as polyalkylene glycols, polyethylene oxide, polypropylene oxide or copolymers thereof with bisglycidyl ethers.

13. Polyacetals such as polyoxymethylene and those polyoxymethylenes which contain ethylene oxide as a comonomer; polyacetals modified with thermoplastic polyurethanes, acrylates or MBS.

14. Polyphenylene oxides and sulfides, and mixtures of polyphenylene oxides with styrene polymers or polyamides.

15. Polyurethanes derived from hydroxyl-terminated polyethers, polyesters or polybutadienes on the one hand and aliphatic or aromatic polyisocyanates on the other, as well as precursors thereof.

16. Polyamides and copolyamides derived from diamines and dicarboxylic acids and/or from aminocarboxylic acids or the corresponding lactams, for example polyamide 4, polyamide 6, polyamide 6/6, 6/10, 6/9, 6/12, 4/6, 12/12, polyamide 11, polyamide 12, aromatic polyamides starting from m-xylene diamine and adipic acid; polyamides prepared from hexamethylenediamine and isophthalic or/and terephthalic acid and with or without an elastomer as modifier, for example poly-2,4,4,-trimethylhexamethylene terephthalamide or poly-m-phenylene isophthalamide; and also block copolymers of the aforementioned polyamides with polyolefins, olefin copolymers, ionomers or chemically bonded or grafted elastomers; or with polyethers, e.g. with polyethylene glycol, polypropylene glycol or polytetramethylene glycol; as well as polyamides or copolyamides modified with EPDM or ABS; and polyamides condensed during processing (RIM polyamide systems).

17. Polyureas, polyimides, polyamide-imides, polyetherimides, polyesterimides, polyhydantoins and polybenzimidazoles.

18. Polyesters derived from dicarboxylic acids and diols and/or from hydroxycarboxylic acids or the corresponding lactones or lactides, for example polyethylene terephthalate, polybutylene terephthalate, poly-1,4-dimethylolcyclohexane terephthalate, polyalkylene naphthalate and polyhydroxybenzoates as well as copolyether esters derived from hydroxyl-terminated polyethers, and also polyesters modified with polycarbonates or MBS. Copolyesters may comprise, for example—but are not limited to—polybutylenesuccinate/terephtalate, polybutyleneadipate/terephthalate, polytetramethyleneadipate/terephthalate, polybutylensuccinate/adipate, polybutylensuccinate/carbonate, poly-3-hydroxybutyrate/octanoate copolymer, poly-3-hydroxybutyrate/hexanoate/decanoate terpolymer. Furthermore, aliphatic polyesters may comprise, for example—but are not limited to—the class of poly(hydroxyalkanoates), in particular, poly(propiolactone), poly(butyrolactone), poly(pivalolactone), poly(valerolactone) and poly(caprolactone), polyethylenesuccinate, polypropylenesuccinate, polybutylenesuccinate, polyhexamethylenesuccinate, polyethyleneadipate, polypropyleneadipate, polybutyleneadipate, polyhexamethyleneadipate, polyethyleneoxalate, polypropyleneoxalate, polybutyleneoxalate, polyhexamethyleneoxalate, polyethylenesebacate, polypropylenesebacate, polybutylenesebacate and polylactic acid (PLA) as well as corresponding polyesters modified with polycarbonates or MBS. The term “polylactic acid (PLA)” designates a homo-polymer of preferably poly-L-lactide and any of its blends or alloys with other polymers; a co-polymer of lactic acid or lactide with other monomers, such as hydroxy-carboxylic acids, like for example glycolic acid, 3-hydroxy-butyric acid, 4-hydroxy-butyric acid, 4-hydroxy-valeric acid, 5-hydroxy-valeric acid, 6-hydroxy-caproic acid and cyclic forms thereof; the terms “lactic acid” or “lactide” include L-lactic acid, D-lactic acid, mixtures and dimers thereof, i.e. L-lactide, D-lactide, meso-lacide and any mixtures thereof.

19. Polycarbonates and polyester carbonates.

20. Polyketones.

21. Polysulfones, polyether sulfones and polyether ketones.

22. Crosslinked polymers derived from aldehydes on the one hand and phenols, ureas and melamines on the other hand, such as phenol/formaldehyde resins, urea/formaldehyde resins and melamine/formaldehyde resins.

23. Drying and non-drying alkyd resins.

24. Unsaturated polyester resins derived from copolyesters of saturated and unsaturated dicarboxylic acids with polyhydric alcohols and vinyl compounds as crosslinking agents, and also halogen-containing modifications thereof of low flammability.

25. Crosslinkable acrylic resins derived from substituted acrylates, for example epoxy acrylates, urethane acrylates or polyester acrylates.

26. Alkyd resins, polyester resins and acrylate resins crosslinked with melamine resins, urea resins, isocyanates, isocyanurates, polyisocyanates or epoxy resins.

27. Crosslinked epoxy resins derived from aliphatic, cycloaliphatic, heterocyclic or aromatic glycidyl compounds, e.g. products of diglycidyl ethers of bisphenol A and bisphenol F, which are crosslinked with customary hardeners such as anhydrides or amines, with or without accelerators.

28. Natural polymers such as cellulose, rubber, gelatin and chemically modified homologous derivatives thereof, for example cellulose acetates, cellulose propionates and cellulose butyrates, or the cellulose ethers such as methyl cellulose; as well as rosins and their derivatives.

29. Blends of the aforementioned polymers (polyblends), for example PP/EPDM, Polyamide/EPDM or ABS, PVC/EVA, PVC/ABS, PVC/MBS, PC/ABS, PBTP/ABS, PC/ASA, PC/PBT, PVC/CPE, PVC/acrylates, POM/thermoplastic PUR, PC/thermoplastic PUR, POM/acrylate, POM/MBS, PPO/HIPS, PPO/PA 6.6 and copolymers, PA/HDPE, PA/PP, PA/PPO, PBT/PC/ABS or PBT/PET/PC.

Preferably component (a) is a thermoplastic organic polymer or a coating binder.

For example the thermoplastic polymer is a polyolefin and the coating binder is an acid catalyzed two component system or an air drying system.

In general, the compounds of the present invention are employed in from about 0.01 to about 5% by weight of the stabilized composition, although this will vary with the particular substrate and application. An advantageous range is from about 0.05 to about 3%, and especially 0.05 to about 1%.

The composition described above may comprise a further component selected from solvents, pigments, dyes, plasticizers, antioxidants, thixotropic agents, levelling assistants, further light stabilizers, metal passivators, metal oxides, organophosphorus compounds, hydroxylamines, UV absorbers, sterically hindered amines, and mixtures thereof.

Examples are given below.

1. Antioxidants

1.1. Alkylated monophenols, for example 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethyl-phenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1-yl)phenol, 2,4-dimethyl-6-(1-methylheptadec-1′-yl)phenol, 2,4-dimethyl-6-(1′-methyltridec-1-yl)phenol and mixtures thereof.

1.2. Alkylthiomethylphenols, for example 2,4-dioctylthiomethyl-6-tert-butylphenol, 2,4-dioctyl-thiomethyl-6-methylphenol, 2,4-dioctylthiomethyl-6-ethylphenol , 2,6-di-dodecylthiomethyl-4-nonylphenol.

1.3. Hydroquinones and alkylated hydroquinones, for example 2,6-di-tert-butyl-4-methoxy-phenol, 2,5-di-tert-butylhydroquinone, 2,5-di-tert-amylhydroquinone, 2,6-diphenyl-4-octade-cyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate.

1.4. Tocopherols, for example α-tocopherol, β-tocopherol, γ-tocopherol, δ-tocopherol and mixtures thereof (vitamin E).

1.5. Hydroxylated thiodiphenyl ethers, for example 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)disulfide.

1.6. Alkylidenebisphenols, for example 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butyl-phenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis(5-tert-butyl-4-hydroxy2-methylphenyl)-4-n-dodecylmercaptobutane, 1,1,5,5-tetra-(5-tert-butyl-4-hydroxy-2-methylphenyl)pentane.

1.7. O-, N- and S-benzyl compounds, for example 3,5,3′,5′-tetra-tert-butyl-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxy-benzyl)sulfide, isooctyl-3,5-di-tert-butyl-4-hydroxybenzylmercaptoacetate.

1.8. Hydroxybenzylated malonates, for example dioctadecyl-2,2-bis(3,5-di-tert-butyl-2-hydroxybenzyl)malonate, di-octadecyl-2-(3-tert-butyl-4-hydroxy-5-methylbenzyl)malonate, di-dodecylmercaptoethyl-2,2-bis (3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate.

1.9. Aromatic hydroxybenzyl compounds, for example 1,3,5-tris(3,5-di-tert-butyl-4-hydroxy-benzyl)-2,4,6-trimethylbenzene, 1,4-bis(3,5-di-tert-butyl-4-hydroxybenzyl)-2,3,5,6-tetramethyl benzene, 2,4,6-tris(3,5-di-tert-butyl-4-hydroxybenzyl)phenol.

1.10. Triazine compounds, for example 2,4-bis(octylmercapto)-6-(3,5-di-tert-butyl-4-hydroxy-anilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris-(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris-(3,5-di-tert-butyl-4-hydroxyphenylethyl)-1,3,5-triazine, 1,3,5-tris(3,5-di-tert-butyl-4-hydroxyphenylpropionyl)-hexahydro-1,3,5-triazine, 1,3,5-tris(3,5-dicyclohexyl-4-hydroxybenzyl)iso-cyanurate.

1.11. Benzylphosphonates, for example dimethyl-2,5-di-tert-butyl-4-hydroxybenzylphosphonate, diethyl-3,5-di-tert-butyl-4-hydroxybenzylphosphonate, dioctadecyl3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid.

1.12. Acylaminophenols, for example 4-hydroxylauranilide, 4-hydroxystearanilide, octyl N-(3,5-di-tert-butyl-4-hydroxyphenyl)carbamate.

1.13. Esters of β-(3,5-di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.14. Esters of β-(5-tert-butyl-4-hydroxy-3-methylphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis-(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane; 3,9-bis[2-{3-(3-tert-butyl-4-hydroxy-5-methylphenyl)propionyloxy}-1,1-dimethylethyl]-2,4,8,10-tetraoxaspiro[5.5]-undecane.

1.15. Esters of β-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, tri-ethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.16. Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N, N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.17. Amides of β-(3,5-di-tert-butyl-4-hydroxyphenyl)propionic acid e.g. N,N′-bis(3,5-di-tert-butyl-4-hydroxyphenylpropionyl)hexamethylenediamide, N,N′-bis(3,5-di-tert-butyl-4-hydroxy-phenylpropionyl)trimethylenediamide, N,N′-bis(3,5-di-tert-butyl-4-hydroxyphenylpropionyphydrazide, N,N′-bis[2-(3-[3,5-di-tert-butyl-4-hydroxyphenyl]propionyloxy)ethyl]oxamide (Naugard®XL-1, supplied by Uniroyal).

1.18. Ascorbic Acid (Vitamin C)

1.19. Aminic antioxidants, for example N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenyl-amine, N-phenyl-1-naphthylamine, N-(4-tert-octylphenyl)-1-naphthylamine, N-phenyl-2-naphthylamine, octylated diphenylamine, for example p,p′-di-tert-octyldiphenylamine, 4-n-butyl-aminophenol, 4-butyrylaminophenol, 4-nonanoylaminophenol, 4-dodecanoylaminophenol, 4-octadecanoylaminophenol, bis(4-methoxyphenyl)amine, 2,6-di-tert-butyl-4-dimethylamino-methylphenol, 2,4′-diaminodiphenylmethane, 4,4′-diaminodiphenylmethane, N,N,N′,N′-tetra-methyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenyl-amines, a mixture of mono- and dialkylated nonyldiphenylamines, a mixture of mono- and dialkylated dodecyldiphenylamines, a mixture of mono- and dialkylated isopropyl/isohexyl-diphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octyl-phenothiazines, N-allylphenothiazine, N,N,N′,N′-tetraphenyl-1,4-diaminobut-2-ene.

2. UV Absorbers and Light Stabilizers

2.1. 2-(2′-Hydroxyphenyl)benzotriazoles, for example 2-(2′-hydroxy-5′-methylphenyl)-benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chloro-benzotriazole, 2-(3′-tert-butyl-2′-hydroxy-5′-methylphenyl)-5-chloro-benzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis-(α,α-dimethylbenzyl)-2′-hydroxyphenyl)benzotriazole, 2-(3′-tert-butyl-2′-hydroxy-5′-(2-octyloxycarbonylethyl)phenyl)-5-chloro-benzotriazole, 2-(3′-tert-butyl-5′-[2-(2-ethylhexyloxy)-carbonylethyl]-2′-hydroxyphenyl)-5-chloro-benzotriazole, 2-(3′-tert-butyl-2′-hydroxy-5′-(2-methoxycarbonylethyl)phenyl)-5-chloro-benzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylene-bis[4-(1,1,3,3-tetramethylbutyl)-6-benzotriazole-2-ylphenol]; the transesterification product of 2-[3′-tert-butyl-5′-(2-methoxycarbonylethyl)-2′-hydroxyphenyl]-2H-benzotriazole with polyethylene glycol 300;

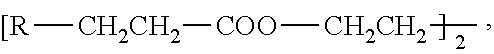

where R=3′-tert-butyl-4′-hydroxy-5′-2H-benzotriazol-2-ylphenyl, 2-[2′-hydroxy-3′-(α,α-dimethylbenzyl)-5′-(1,1,3,3-tetramethylbutyl)-phenyl]-benzotriazole; 2-[2′-hydroxy-3′-(1,1,3,3-tetramethylbutyl)-5′-(α,α-dimethylbenzyl)-phenyl]benzotriazole.

2.2. 2-Hydroxybenzophenones, for example the 4-hydroxy, 4-methoxy, 4-octyloxy, 4-decyloxy, 4-dodecyloxy, 4-benzyloxy, 4,2′,4′-trihydroxy and 2′-hydroxy-4,4′-dimethoxy derivatives.

2.3. Esters of substituted and unsubstituted benzoic acids, for example 4-tert-butyl-phenyl salicylate, phenyl salicylate, octylphenyl salicylate, dibenzoyl resorcinol, bis(4-tert-butylbenzoyl)resorcinol, benzoyl resorcinol, 2,4-di-tert-butylphenyl 3,5-di-tert-butyl-4-hydroxybenzoate, hexadecyl 3,5-di-tert-butyl-4-hydroxybenzoate, octadecyl 3,5-di-tert-butyl-4-hydroxybenzoate, 2-methyl-4,6-di-tert-butylphenyl 3,5-di-tert-butyl-4-hydroxybenzoate.

2.4. Acrylates, for example ethyl α-cyano-β,β-diphenylacrylate, isooctyl α-cyano-β,β-diphenylacrylate, methyl α-carbomethoxycinnamate, methyl α-cyano-β-methyl-p-methoxycinnamate, butyl α-cyano-β-methyl-p-methoxy-cinnamate, methyl α-carbomethoxy-p-methoxycinnamate, N-(β-carbomethoxy-β-cyanovinyl)-2-methylindoline, neopentyl tetra(α-cyano-β,β-di-phenylacrylate.

2.5. Nickel compounds, for example nickel complexes of 2,2′-thio-bis[4-(1,1,3,3-tetramethyl-butyl)phenol], such as the 1:1 or 1:2 complex, with or without additional ligands such as n-butylamine, triethanolamine or N-cyclohexyldiethanolamine, nickel dibutyldithiocarbamate, nickel salts of the monoalkyl esters, e.g. the methyl or ethyl ester, of 4-hydroxy-3,5-di-tert-butylbenzylphosphonic acid, nickel complexes of ketoximes, e.g. of 2-hydroxy-4-methylphenylundecylketoxime, nickel complexes of 1-phenyl-4-lauroyl-5-hydroxypyrazole, with or without additional ligands.

2.6. Sterically hindered amines, for example bis(2,2,6,6-tetramethyl-4-piperidyl)sebacate, bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl) n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butanetetracarboxylate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl)malonate, 3-n-octyl-7,7,9,9-tetramethyl-1,3,8-triazaspiro[4.5]decane-2,4-dione, bis(1-octyloxy-2,2,6,6-tetramethylpiperidyl)sebacate, bis(1-octyloxy-2,2,6,6-tetramethylpiperidyl)succinate, linear or cyclic condensates of N,N′-bis(2,2,6,6-tetramethyl-4-piperidyl)hexamethylenediamine and 4-morpholino-2,6-dichloro-1,3,5-triazine, the condensate of 2-chloro-4,6-bis(4-n-butylamino-2,2,6,6-tetramethylpiperidyl)-1,3,5-triazine and 1,2-bis(3-aminopropylamino)ethane, the condensate of 2-chloro-4,6-di-(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)pyrrolidine-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, a condensate of N,N′-bis(2,2,6,6-tetramethyl-4-piperidyl)hexamethylenediamine and 4-cyclohexylamino-2,6-dichloro-1,3,5-triazine, a condensate of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); a condensate of 1,6-hexanediamine and 2,4,6-trichloro-1,3,5-triazine as well as N,N-dibutylamine and 4-butylamino-2,2,6,6-tetramethylpiperidine (CAS Reg. No. [192268-64-7]); N-(2,2,6,6-tetramethyl-4-piperidyl)-n-dodecylsuccinimide, N-(1,2,2,6,6-pentamethyl-4-piperidyl)-n-dodecylsuccinimide, 2-undecyl-7,7,9,9-tetramethyl-1-oxa-3,8-diaza-4-oxo-spiro[4,5]decane, a reaction product of 7,7,9,9-tetramethyl-2-cycloundecyl-1-oxa-3,8-diaza-4-oxospiro-[4,5]decane and epichlorohydrin, 1,1-bis(1,2,2,6,6-pentamethyl-4-piperidyloxycarbonyl)-2-(4-methoxyphenypethene, N,N′-bis-formyl-N,N′-bis(2,2,6,6-tetramethyl-4-piperidyl)hexamethylenediamine, a diester of 4-methoxymethylenemalonic acid with 1,2,2,6,6-pentamethyl-4-hydroxypiperidine, poly[methylpropyl-3-oxy-4-(2,2,6,6-tetramethyl-4-piperidyl)]siloxane, a reaction product of maleic acid anhydride-α-olefin copolymer with 2,2,6,6-tetramethyl-4-aminopiperidine or 1,2,2,6,6-pentamethyl-4-aminopiperidine, 2,4-bis[N-(1-cyclohexyloxy-2,2,6,6-tetramethylpiperidine-4-yl)-N-butylamino]-6-(2-hydroxyethyl)amino-1,3,5-triazine, 1-(2-hydroxy-2-methylpropoxy)-4-octadecanoyloxy-2,2,6,6-tetramethylpiperidine, 5-(2-ethylhexanoyl)-oxymethyl-3,3,5-trimethyl-2-morpholinone, Sanduvor (Clariant; CAS Reg. No. 106917-31-1],5-(2-ethylhexanoyl)oxymethyl-3,3,5-trimethyl-2-morpholinone, the reaction product of 2,4-bis-[(1-cyclohexyloxy-2,2,6,6-piperidine-4-yl)butylamino]-6-chloro-s-triazine with N,N′-bis(3-aminopropyl)ethylenediamine), 1,3,5-tris(N-cyclohexyl-N-(2,2,6,6-tetramethylpiperazine-3-one-4-yl)amino)-s-triazine, 1,3,5-tris(N-cyclohexyl-N-(1,2,2,6,6-pentamethylpiperazine-3-one-4-yl)-amino)-s-triazine.

2.7. Oxamides, for example 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy-disubstituted oxanilides and mixtures of o- and p-ethoxy-disubstituted oxanilides.

2.8. 2-(2-Hydroxyphenyl)-1,3,5-triazines, for example 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropyloxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine, 2,4-bis(4-[2-ethylhexyloxy]-2-hydroxyphenyl)-6-(4-methoxyphenyl)-1,3,5-triazine.

3. Metal deactivators, for example N,N′-diphenyloxamide, N-salicylal-N′-salicyloyl hydrazine, N,N′-bis(salicyloyl)hydrazine, N,N′-bis(3,5-di-tert-butyl-4-hydroxyphenylpropionyl)hydrazine, 3-salicyloylamino-1,2,4-triazole, bis(benzylidene)oxalyl dihydrazide, oxanilide, isophthaloyl dihydrazide, sebacoyl bisphenylhydrazide, N,N′-diacetyladipoyl dihydrazide, N,N′-bis(salicyloyl)oxalyl dihydrazide, N,N′-bis(salicyloyl)thiopropionyl dihydrazide.

4. Phosphites and phosphonites, for example triphenyl phosphite, diphenylalkyl phosphites, phenyldialkyl phosphites, tris(nonylphenyl) phosphite, trilauryl phosphite, trioctadecyl phosphite, distearylpentaerythritol diphosphite, tris(2,4-di-tert-butylphenyl) phosphite, diisodecyl pentaerythritol diphosphite, bis(2,4-di-tert-butylphenyl)pentaerythritol diphosphite, bis(2,4-dicumylphenyl)pentaerythritol diphosphite, bis(2,6-di-tert-butyl-4-methylphenyl)pentaerythritol diphosphite, diisodecyloxypentaerythritol diphosphite, bis(2,4-di-tert-butyl-6-methylphenyl)pentaerythritol diphosphite, bis(2,4,6-tris(tert-butylphenyl)pentaerythritol diphosphite, tristearyl sorbitol triphosphite, tetrakis(2,4-di-tert-butylphenyl) 4,4′-biphenylene diphosphonite, 6-isooctyloxy-2,4,8,10-tetra-tert-butyl-12H-dibenz[d,g]-1,3,2-dioxaphosphocin, bis(2,4-di-tert-butyl-6-methylphenyl)methyl phosphite, bis(2,4-di-tert-butyl-6-methylphenyl)ethyl phosphite, 6-fluoro-2,4,8,10-tetra-tert-butyl-12-methyl-dibenz[d,g]-1,3,2-dioxaphosphocin, 2,2′,2″-nitrilo-[triethyltris(3,3′,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-diyl)phosphite, 5-butyl-5-ethyl-2-(2,4,6-tri-tert-butylphenoxy)-1,3,2-dioxaphosphirane.

The following phosphites are especially preferred:

Tris(2,4-di-tert-butylphenyl) phosphite (Irgafos®168, Ciba Specialty Chemicals Inc.), tris(nonylphenyl) phosphite,

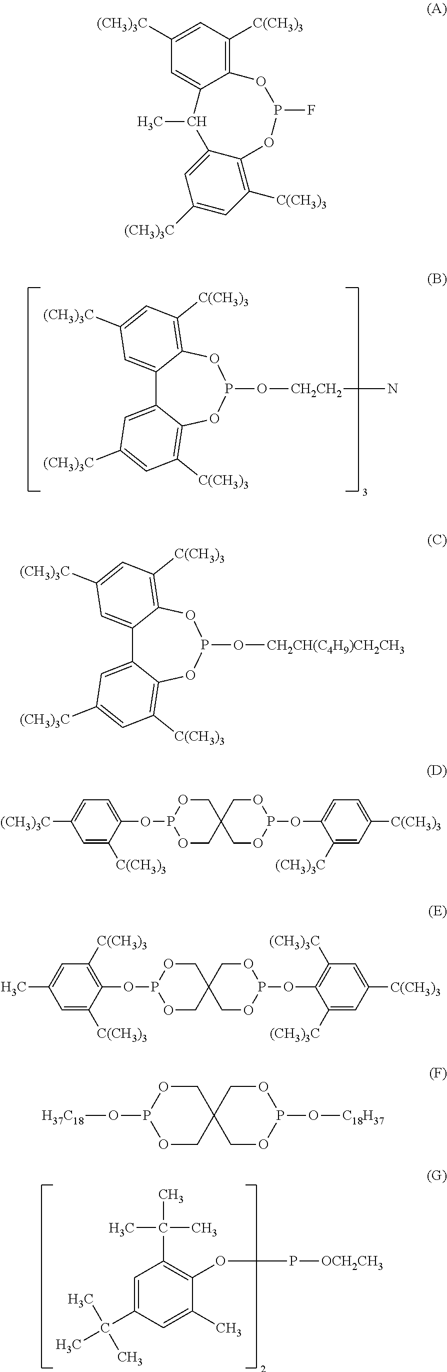

5. Hydroxylamines, for example N,N-dibenzylhydroxylamine, N,N-diethylhydroxylamine, N,N-dioctylhydroxylamine, N,N-dilaurylhydroxylamine, N,N-ditetradecylhydroxylamine, N,N-dihexadecylhydroxylamine, N,N-dioctadecylhydroxylamine, N-hexadecyl-N-octadecylhydroxylamine, N-heptadecyl-N-octadecylhydroxylamine, N,N-dialkylhydroxylamine derived from hydrogenated tallow amine.

6. Nitrones, for example, N-benzyl-alpha-phenylnitrone, N-ethyl-alpha-methylnitrone, N-octyl-alpha-heptylnitrone, N-lauryl-alpha-undecylnitrone, N-tetradecyl-alpha-tridecylnnitrone, N-hexadecyl-alpha-pentadecylnitrone, N-octadecyl-alpha-heptadecylnitrone, N-hexadecyl-alpha-heptadecylnitrone, N-ocatadecyl-alpha-pentadecylnitrone, N-heptadecyl-alpha-hepta-decylnitrone, N-octadecyl-alpha-hexadecylnitrone, nitrone derived from N,N-dialkylhydroxyl-amine derived from hydrogenated tallow amine.

7. Thiosvnergists, for example dilauryl thiodipropionate, dimistryl thiodipropionate, distearyl thiodipropionate or distearyl disulfide.

8. Peroxide scavengers, for example esters of β-thiodipropionic acid, for example the lauryl, stearyl, myristyl or tridecyl esters, mercaptobenzimidazole or the zinc salt of 2-mercapto-benzimidazole, zinc dibutyldithiocarbamate, dioctadecyl disulfide, pentaerythritol tetrakis(β-dodecylmercapto)propionate.

9. Polyamide stabilizers, for example copper salts in combination with iodides and/or phosphorus compounds and salts of divalent manganese.

10. Basic co-stabilizers, for example melamine, polyvinylpyrrolidone, dicyandiamide, triallyl cyanurate, urea derivatives, hydrazine derivatives, amines, polyamides, polyurethanes, alkali metal salts and alkaline earth metal salts of higher fatty acids, for example calcium stearate, zinc stearate, magnesium behenate, magnesium stearate, sodium ricinoleate and potassium palmitate, antimony pyrocatecholate or zinc pyrocatecholate.

11. Nucleating agents, for example inorganic substances, such as talcum, metal oxides, such as titanium dioxide or magnesium oxide, phosphates, carbonates or sulfates of, preferably, alkaline earth metals; organic compounds, such as mono- or polycarboxylic acids and the salts thereof, e.g. 4-tert-butylbenzoic acid, adipic acid, diphenylacetic acid, sodium succinate or sodium benzoate; polymeric compounds, such as ionic copolymers (ionomers). Especially preferred are 1,3:2,4-bis(3′,4′-dimethylbenzylidene)sorbitol, 1,3:2,4-di(paramethyldibenzylidene)sorbitol, and 1,3:2,4-di(benzylidene)sorbitol.

12. Fillers and reinforcing agents, for example calcium carbonate, silicates, glass fibres, glass beads, asbestos, talc, kaolin, mica, barium sulfate, metal oxides and hydroxides, carbon black, graphite, wood flour and flours or fibers of other natural products, synthetic fibers.

13. Other additives, for example plasticisers, lubricants, emulsifiers, pigments, rheology additives, catalysts, flow-control agents, optical brighteners, flameproofing agents, antistatic agents and blowing agents.

14. Benzofuranones and indolinones, for example those disclosed in U.S. Pat. No. 4,325,863; U.S. Pat. No. 4,338,244; U.S. Pat. No. 5,175,312; U.S. Pat. No. 5,216,052; U.S. Pat. No. 5,252,643; DE-A-4316611; DE-A-4316622; DE-A-4316876; EP-A-0589839, EP-A-0591102; EP-A-1291384 or 3-[4-(2-acetoxyethoxy)phenyl]-5,7-di-tert-butylbenzofuran-2-one, 5,7-di-tert-butyl-3-[4-(2-stearoyloxy-ethoxy)phenyl]benzofuran-2-one, 3,3′-bis[5,7-d i-tert-butyl-3-(4-[2-hydroxyethoxy]phenyl)benzofuran-2-one], 5,7-di-tert-butyl-3-(4-ethoxyphenyl)benzofuran-2-one, 3-(4-acetoxy-3,5-di-methylphenyl)-5,7-di-tert-butylbenzofuran-2-one, 3-(3,5-dimethyl-4-pivaloyloxyphenyl)-5,7-di-tert-butylbenzofuran-2-one, 3-(3,4-dimethylphenyl)-5,7-di-tert-butylbenzofuran-2-one, 3-(2,3-dimethylphenyl)-5,7-di-tert-butylbenzofuran-2-one, 3-(2-acetyl-5-isooctylphenyl)-5-isooctyl-benzofuran-2-one.

The stabilizers of the instant invention may readily be incorporated into the organic polymers by conventional techniques, at any convenient stage prior to the manufacture of shaped articles therefrom. For example, the stabilizer may be mixed with the polymer in dry powder form, or a suspension or emulsion of the stabilizer may be mixed with a solution, suspension, or emulsion of the polymer. The resulting stabilized compositions of the invention may optionally also contain from about 0.01 to about 5%, preferably from about 0.025 to about 2%, and especially from about 0.1 to about 1% by weight of various conventional additives, such as the materials listed above, or mixtures thereof.

Yet further aspects of the invention are a process for stabilizing an organic polymeric material against damage by light, oxygen and/or heat, which comprises adding to or applying to said material at least one compound according to formula (I), of formula (I′) or of formulae 1 to 16 as described above; and

the use of a compound according to formula (I), of formula (I′) or of formulae 1 to 16 as described above for stabilizing an organic polymer against damage by light, oxygen and/or heat or as flame retardant.

The definitions and preferences outlined for the compounds apply equally to the other aspects of the invention.

The following examples illustrate the invention.

Step 1: Formation of NOR Precursors

4-hydroxy NORs and 4-oxo-NORs were prepared according to WO 2008003602.

Step 2: Epoxy NORs

The precursors are prepared according to the reaction scheme below.

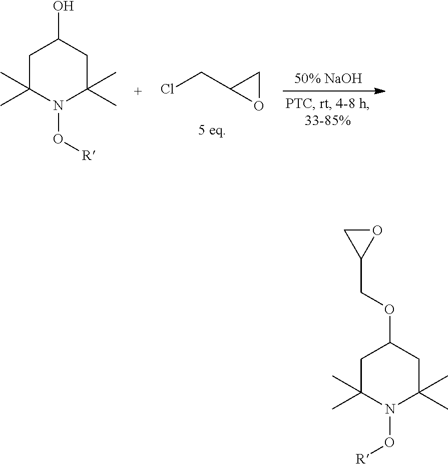

### EXAMPLE 1

### 1-Methoxy-2,2,6,6-tetramethyl-4-oxiranylmethoxy-piperidine

In a 250 ml round bottom flask, 100 g 50% NaOH, 0.5 g NBu₄Br and 45 g epichlorohydrin are mixed. 19.0 g 1-Methoxy-2,2,6,6-tetramethylpiperidin-4-ol are added in small portions over a period of 30 min. After stirring at room temperature over night, the reaction mixture is diluted with 200 ml H₂O. The product is extracted with ethyl acetate twice and the combined organic phases are consecutively washed with H₂O, 5% NaHSO₄ solution and brine. The organic solvent is removed in vacuo, and the crude product is filtered over a pad of silica gel (hexan/acetone 9:1) to provide, after removal of the solvent, 19.4 g of clear, pale yellow oil. MS: m/z=244 [M+H]⁺.

Examples 2-7 are prepared in analogy to Example 1

| Example | 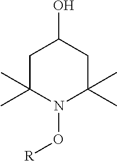 R = | 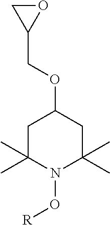 R = | Yield [%] |
|---|---|---|---|
| 2 | ethyl | ethyl | 80 |
| 3 | propyl | propyl | 85 |
| 4 | cyclohexyl | cyclohexyl | 75 |
| 5 | octyl | octyl | 54 |
| 6 | undecyl | undecyl | 37 |
| 7 | CH₂C(CH₃)₂OH | CH₂C(CH₃)₂OH + | 63 |
| | | (bis epoxide) | 15 |

### EXAMPLE 8

### (1-Propoxy-2,2,6,6-tetramethyl-piperidin-4-yl)-oxiranylmethyl-amine

37.4 g 1-methoxy-2,2,6,6-tetramethyl-piperidin-4-one are dissolved in 200 ml methanol and 14.0 g n-butylamine and 10 g sodium sulfate are added. The mixture is stirred over night at rt. The mixture is filtered and transferred into a stainless steel autoclave. 0.5 g 10% palladium on charcoal are added and the autoclav is pressurized with 5 bar of hydrogen. The reaction mixture is stirred at 60-75° C. for 20 h. The reaction mixture is then filtered over celite and the methanol is removed in vacuo to give 42.2 g of a yellowish solid. MS: m/z=244 [M+H]⁺. To 150 ml toluene are added 20 g butyl-(1-propoxy-2,2,6,6-tetramethyl-piperidin-4-yl)-amine, 15.5 g epichlorohydrin, 100 mg Aliquat® 336 and 35 g 50% NaOH. The reaction mixture is stirred at 65° C. for 16 h. After separation of the aqueous and the organic phase, the latter one is thoroughly washed with H₂O. After drying over Na₂SO₄, the mixture is treated with 1 g char coal at rt. The coal is removed by filtration and the volatile organic compounds are removed under vacuum to afford 20.4 g of 1-propoxy-2,2,6,6-tetramethyl-piperidin-4-yl)oxiranylmethyl-amine; yellow oil, MS: m/z=299 [M+H]⁺.

### EXAMPLE 10

### 9-Propoxy-3,8,8,10,10-pentamethyl-3-oxiranylmethoxymethyl-1,5-dioxa-9-aza-spiro[5.5]undecane

50 g 1-Propoxy-2,2,6,6-tetramethyl-piperidin-4-one, 29 g 2-hydroxymethyl-2-methyl-1,3-propanediol and 0.5 g p-toluene sulfonic acid are added to 250 ml toluene. The mixture is heated to reflux while removing water in a Dean-Stark-trap. After disappearance of the ketone, the mixture is washed with H₂O, dryed over Na₂SO₄ and filtered over a bed of silica gel. After removal of the solvent, 68.9 g of (3,8,8,10,10-pentamethyl-9-propoxy-1,5-dioxa-9-aza-spiro[5.5]undec-3-yl)-methanol are obtained; MS: m/z=316 [M+H]⁺.

20 g (3,8,8,10,10-pentamethyl-9-propoxy-1,5-dioxa-9-aza-spiro[5.5]undec-3-yl)-methanol are transformed into the corresponding epoxide following example 1. Yield: 16.4 g, pale yellow oil. MS: m/z=372 [M+H]⁺.

### EXAMPLE 11

### 8-Methoxy-2,2,7,7,9,9-hexamethyl-3-oxiranylmethyl-1-oxa-3,8-diaza-spiro[4.5] decan-4-one

To a soln. of 3.26 g 1-methoxy-2,2,6,6-tetramethyl-piperidin-4-one in 40 ml of dichloromethane is added 2.09 g trimethylsilyl cyanide and 0.28 g tetrabutylammonium-1-hydroxyphthalimide. After 2 h of stirring at rt, the ketone is completely converted. The reaction mixture is diluted with 90 ml ethyl acetate and 20 ml hexane. After washing with water and drying over sodium sulfate, the organic solvents are removed in vacuo to afford 4.7 g of 1-methoxy-2,2,6,6-tetramethyl-4-cyano-4-trimethylsilyloxypiperidine (yellow oil); MS: m/z=285 [M+H]⁺.

1.0 g of this intermediate is dissolved in 1.76 g acetic acid and 0.48 g acetone. 0.7 g Sulfuric acid are added and the reaction mixture is stirred at 70° C. for 18 h while forming a deep red solution. The reaction mixture is added to 40 ml saturated NaHCO₃ solution, and extracted with 40 ml ethyl acetate. The organic phase is washed with H₂O and subsequently dried over sodium sulfate. The solvent is removed in vacuo to leave 0.87 g of a tan residue. After washing the residue with n-hexane, 8-Methoxy-2,2,7,7,9,9-hexamethyl-1-oxa-3,8-diaza-spiro[4.5]decan-4-one is obtained as a white solid (0.46g). MS: m/z=271 [M+H]⁺.

0.25 g 8-Methoxy-2,2,7,7,9,9-hexamethyl-1-oxa-3,8-diaza-spiro[4.5]decan-4-one were transformed into the corresponding epoxide following example 8. Yield: 0.30 g, pale yellow solid. MS: m/z=327 [M+H]⁺.

Step 3: Reaction of Epoxy NORs with HALS Backbones

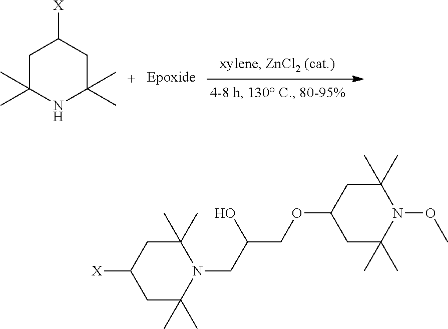

### EXAMPLE 12

### Reaction of Dastib 845 with methoxy-2,2,6,6-tetramethyl-4-oxiranylmethoxy-piperidine

27.3 g 2,2,6,6 -tetramethyl-4-piperidinol fatty acids ester (Dastib 845) and 16.8 g 1-methoxy-2,2,6,6-tetramethyl-4-oxiranylmethoxy-piperidine are dissolved in 90 ml xylene and 100 mg ZnCl₂ are added. The reaction mixture is heated to 130° C. for 5 h. Upon cooling to rt, the reaction mixture is washed with water, dried over Na₂SO₄ and treated with 1 g char coal. The coal is removed by filtration, and the solvent is removed under vacuum to afford a yellowish oil with solidified into a beige wax. The crude product is dissolved in hot ethanol and, upon cooling, a white precipitate formed. Yield: 97%, mp 47-49° C., MS: m/z=667 (ca. 37%), 653 (ca. 57%), 638 (ca. 3%) [M+H]⁺.

### EXAMPLE 13

### Reaction of Chimassorb 944 with 1-ethoxy-2,2,6,6-tetramethyl-4-oxiranyl-methoxy-piperidine

22.8 g Chimassorb 944 and 26.3 g 1-ethoxy-2,2,6,6-tetramethyl-4-oxiranylmethoxy-piperidine are dissolved in 150 ml xylene and 500 mg ZnCl₂ are added. The reaction mixture is heated to 140° C. for 12 h. Upon cooling to rt, the reaction mixture is washed with water, dried over Na₂SO₄ and treated with 1 g char coal. The coal is removed by filtration, and the solvent is removed under vacuum to afford 42 g of an amorphous yellow solid. Anal. calcd. C, 68.17; H, 11.11; N, 11.87. Found: C, 68.15; H, 11.23; N, 11.71

The following compounds have been prepared accordingly.

| Ex- | | | |
|---|---|---|---|
| ample | HALS | Product | yield |
| 14 | Tetramethyl piperidine | 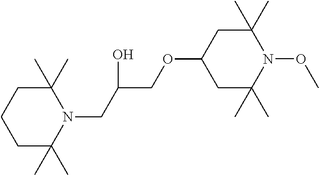 | 85%, solid MS: m/z = 385 [M + H]⁺. |
| 15 | Malonic acid bis-(2,2,6,6- tetramethyl- piperidin- 4-yl)ester | 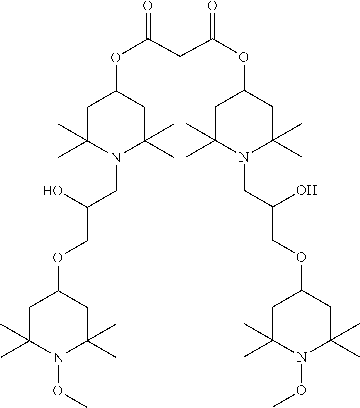 | 93%, vicous oil MS: m/z = 869 [M + H]⁺. |
| 16 | N,N′-Bis (2,2,6,6- tetramethyl- 4- piperidinyl)- 1,6-hexane- diamine | 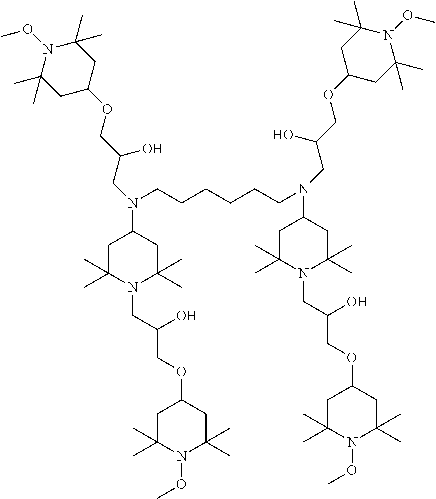 | 77%, viscous oil MS: m/z = 1367 [M + H]⁺. |
| 17 | 2,2,6.6- tetramethyl- N-(2,2,6,6- tetramethyl- 4-piper- idinyl)-4- piper- idinamine | 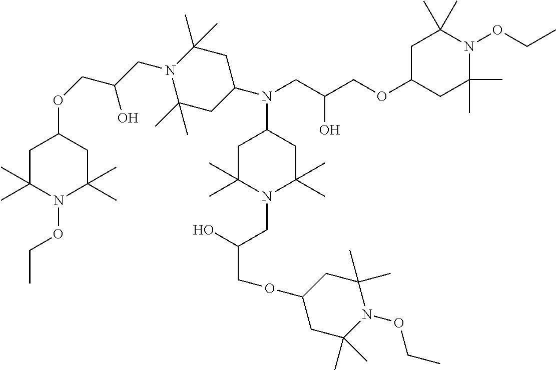 | 66%, vicous oil MS: m/z = 1067 [M + H]⁺. |
| 18 | Tinuvin 770 | 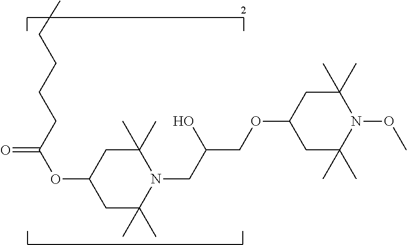 | 84%, viscous oil MS: m/z = 967 [M + H]⁺. |
| 19 | 2,2,4,4, 14,14,16,16- octamethyl- 7,11,18,21- tetraoxa-3,15- diazatrispiro [5.2.2.5.2.2] heneicosane | 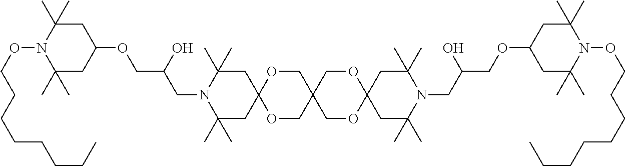 | 27%, beige solid MS: m/z = 1093 [M + H]⁺. |
| 20 | Bis(2,2,6,6- tetramethyl-4- piperidinyl) carbonate | 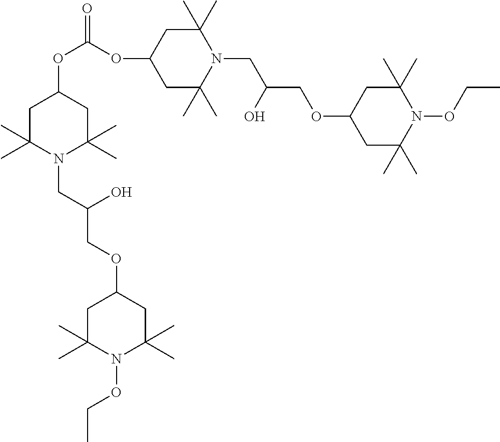 | 76%, semi- solid MS: m/z = 855 [M + H]⁺. |
| 21 | Dastib 845 | 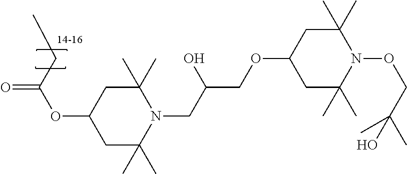 | 91%, solid MS: m/z = 725, 710, 695 [M + H]⁺. |
| 22 | N-Dodecyl- 2,2,6,6- tetramethyl- piperidin- 4-amine | 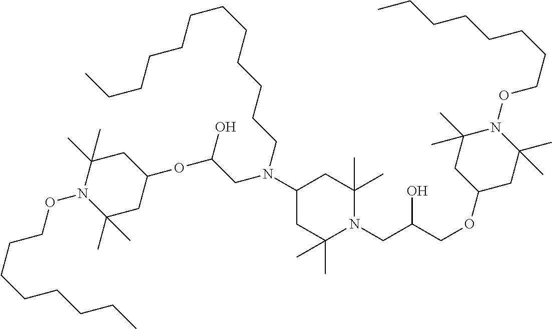 | 85%, wax MS: m/z = 993 [M + H]⁺. |
| 23 | Tinuvin 770 | 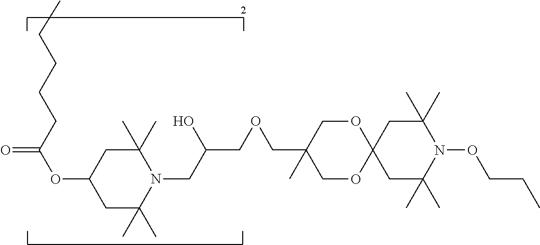 | 83%, semi- solid MS: m/z = 1223 [M + H]⁺. |
| 24 | Hostavin 3055 | 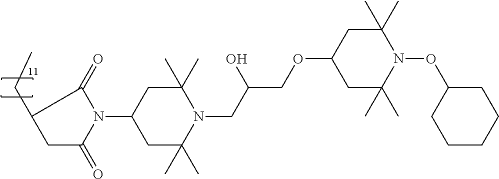 | 85%, wax, MS: m/z = 718 [M + H]⁺. |
| 25 | Dastib 845 | 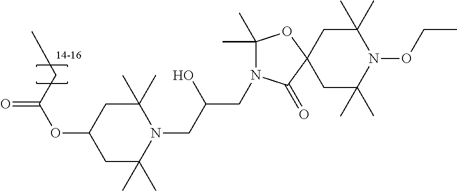 | 77%, solid. MS: m/z = 764, 749, 735 [M + H]⁺. |
| 26 | Dastib 845 | 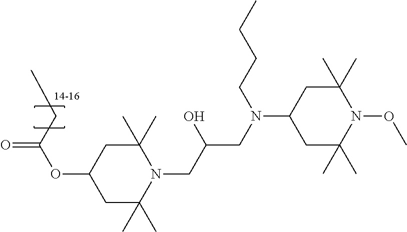 | 87%, solid, MS: m/z = 721, 707, 693 [M + H]⁺ |
| 27 | 4- (Butylamino)- 2,2,6,6- tetramethyl- piperidine | 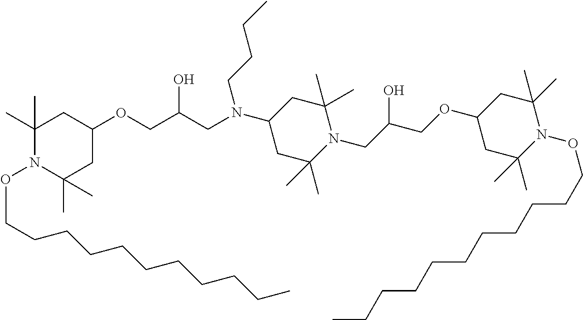 | 83%, wax MS: m/z = 979 [M + H]⁺. |
| 28 | Chimassorb 2020 | 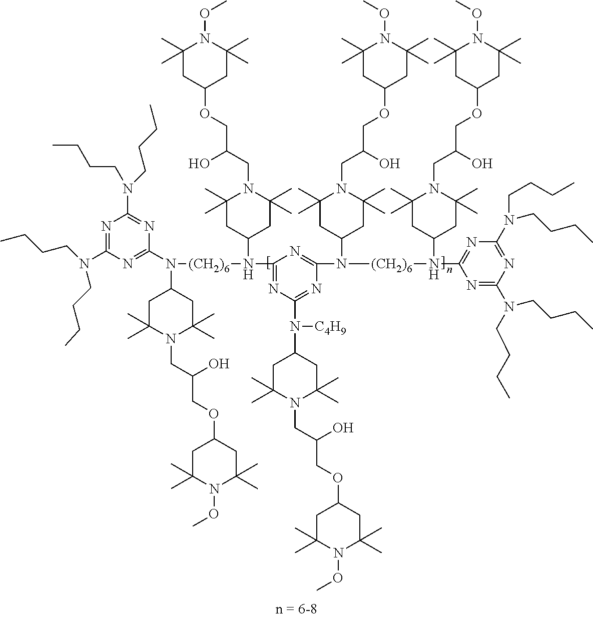 | 89%, solid, Anal. calcd. C, 65.21; H, 10.89; N, 13.64. Found: C, 66.75; H, 10.49; N, 10.01 |
| 29 | Chimassorb 966 (chloro- benzene used as reaction solvent) | 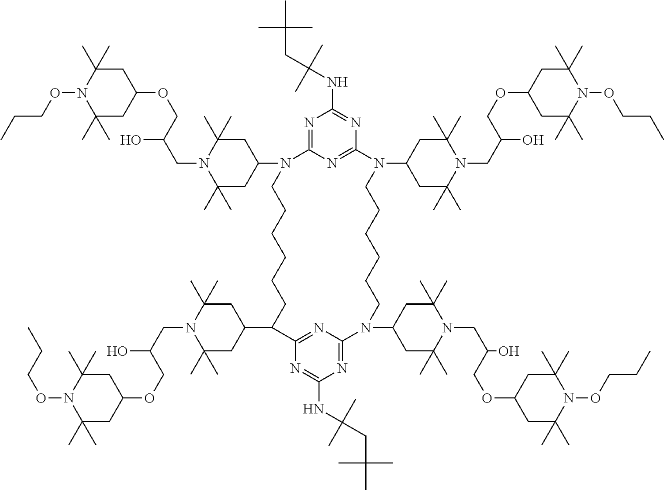 | 97%, solid, MS: m/z = 2281 [M + H]⁺. |
| 30 | Cyasorb 3346 | 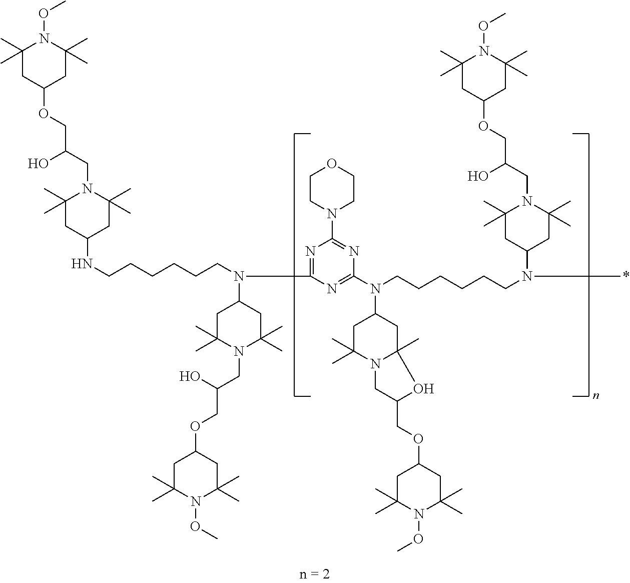 | 56%, brown solid; Anal. calcd. C, 66.31; H, 10.65; N, 12.26. Found: C, 66.73; H, 10.49; N, 12.31. |
| 31 | T7 | 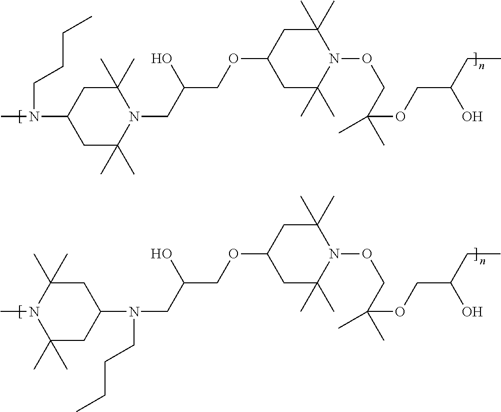 n = 2-8 | 64%, wax, Anal. calcd. C, 66.65; H, 11.53; N, 7.17. Found: C, 66.75; H, 11.49; N, 7.01. |
| 32 | LA-57 | 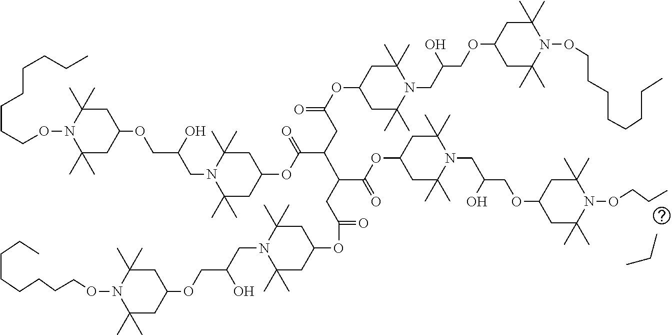 | 41%, wax, MS: m/z = 2155, 1814, 1675, 1473, 1334, 1195, [M + H]⁺. Anal. calcd. C, 69.04; H, 10.93; N, 5.19. Found: C, 68.23; H, 10.49; N, 5.18. |
| 33 | Uvinul 4050 | 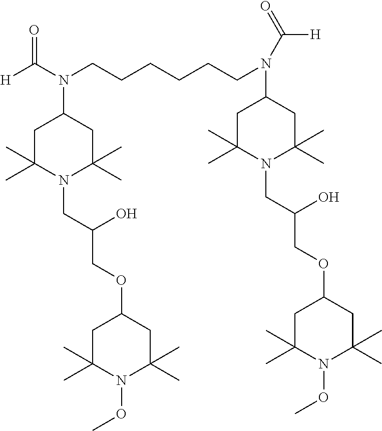 | 91%, solid MS: m/z = 937 [M + H]⁺. |
| 34 | Tetramethyl piperidine | 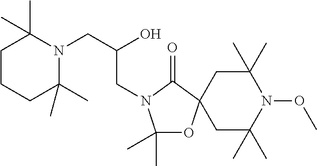 | 87%, solid, MS: m/z = 468 [M + H]⁺. |
| 35 | Tetramethyl piperidine | 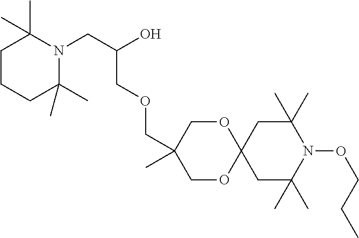 | 82%, solid MS: m/z = 513 [M + H]⁺. |

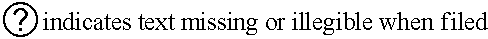

### APPLICATION EXAMPLES B

### Example B1

### Use as Flame Retardant Additives in PP Films

Polypropylene (Moplen® HF500 N) is extruded on a co-rotating twin-screw extruder ZSK18 (Coperion Werner & Pfleiderer) at a temperature of Tmax=190° C. (heating zones 1-7), a throughput rate of 1 kg/h and 100 rpm with the addition of a basic-level stabilization (0.3% IRGANOX B225+0.05% Ca-stearate, IRGANOX B225 is a 1:1 mixture of IRGAFOS 168 and IRGANOX 1010) and 0.5 weight % of the compound of example 33. After cooling in a water bath, the polymer strand is granulated. Test specimens are prepared by compression molding (films 190×90 mm, thickness=0.2 mm or 1.0 mm; Fontune TP200, 230° C.). Test films are tested under DIN 4102-1 B2 test conditions. The results are presented in Table B1

TABLE B1

| Compound | Burning time [s] | Damaged length [mm] |
|---|---|---|
| Blank | 38.3 | 190 |
| Compound of example 33 | 14.0 | 55 |

DIN 4102 - B2 (Edge Ignition, Flame length 40 mm, Distance 16 mm). PP Film Thickness 200 microns; Length: 190 mm; Width: 90 mm; Conditioning Procedure: 3 days 50%/23° C. in conditioning chamber; Lab. humidity 50%/Temp: 23° C.

### Example B2

### Light Stabilization of Polypropylene

A polypropylene basis formulation containing PP EE 013 AE (Borealis), carbon black master batch FK Schwarz 34-270/TPO, talk powder Luzenac A-20 (Luzenac), Irganox B 215 FF (Ciba/BASF), and Ca-stearate (weight % composition based on the weight of the total composition, according to table B2) is compounded in a twin-screw extruder (25 mm) at 220° C. and subsequently granulated.

TABLE B2

| Ingredients | Weight Percent |
|---|---|
| Polypropylene PP EE 013 | 78.40 |
| FK Schawarz 34-270/TPO | 1.50 |
| Luzenac A-20 | 20.000 |
| Irganox B215 FF | 0.05 |
| Ca-Stearate BP | 0.05 |

50 g of this compound and 50 mg each of the additives of the examples 12, 15, 16 are kneaded in a brabender under nitrogen at 200° C. for 10 min. The resulting melt is pressed at 230° C. to yield plaques of 1 mm thickness. Test specimens (20*60 mm) are produced and exposed to light-induced ageing according to Fakra conditions (lightfastness under high temperature conditions, DIN 75202, PSA and SAEJ 2412, accelerated exposure of automotive interior components using a controlled irradiance xenon-arc apparatus). To determine the light stabilization efficiency of the additives, the gloss of the sample specimens is measured at an angle of 85° and the color difference delta E upon light exposure. The data in table B3 indicate the time of failure, i.e. the period after which a delta E>2 and a gloss reduction of 50% of the starting value is measured.

TABLE B3

| | Compound of | Compound of | Compound of |
|---|---|---|---|
| | example | example | example |
| | 12 - time to | 15 - time to | 16 - time to |
| | failure in h | failure in h | failure in h |
| PSA (50% of initial | >3000 | 3000 | >3000 |
| gloss) | | | |
| PSA dE > 2 | 300 | 2000 | 2500 |
| Fakra (50% of initial | >2500 | >2500 | >2500 |
| gloss) | | | |
| Fakra dE > 2 | 2500 | 2500 | 2000 |
| SAE J (50% of initial | >4500 | >3000 | >3000 |
| gloss) | | | |
| SAE J dE > 2 | 4500 | 3000 | 3000 |

### Example B3

### Light Stabilization of Polyethylene Films

### Film Manufacture:

In a turbo mixer (Caccia, Labo 10) additives are mixed with low density polyethylene, LDPE. The mixture is extruded at a maximum temperature of 200° C. using an O.M.C. twin-screw extruder (model ebv 19/25) to granules. The granules are subsequently mixed and diluted with the same LDPE in order to obtain the final composition for preparing a 150 μm thick film, using a blow-extruder (Dolci (RTM)) working at a maximum temperature of 210° C. The final concentrations of the LDPE films are indicated in Table B4.

TABLE B4
Final composition of the LDPE films

| Ref. | Additives |
|---|---|
| Film 1^{a)} | 0.4% Compound 29; 0.1% Irganox 1010^{c)} |
| Film 2^{a)} | 0.4% compound 13; 0.1% Irganox 1010^{c)} |
| Film 3^{b)} | 0.4% Uvinul 5050H^{e)} (commercial product as |
| | reference); 0.1% Irganox 1010^{c)} |

^{a)}according to the invention
^{b)}comparative
^{c)}Irganox 1010 (RTM, Ciba) is tetrakis-[3-(3,5-di-tert-butyl-4-hydroxy-phenyl)-propionyl-oxymethyl]-methane
^{d)}Tinuvin NOR 371 (RTM, BASF) is a mixture of oligomeric compounds which are the formal condensation products of N,N′-bis-(2,2,6,6-tetramethyl-1-propoxy-piperidin-4-yl)-hexane-1,6-diamine and 2,4-dichloro-6-{n-butyl-(2,2,6,6-tetramethyl-1-propoxy-piperidin-4-yl)-amino}-[1,3,5]triazine end-capped with 2-chloro-4,6-bis-(di-n-butylamino)-[1,3,5]triazine
^{e)}Uvinul 5050H (RTM, BASF) is a mixture of oligomeric compounds which are the formal condensation product of 4-Amino-2,2,6,6-Tetramethylpiperidine and Octadecene-1-Maleic Anhydride Copolymer

### Example B4

### Light Stabilization of Polyethylene Films

Light exposure: LDPE films are exposed in an ATLAS Weatherometer (model Ci65A) equipped with a 6500W Xenon lamp (0.35 W/m²; continuous light cycle, black panel temperature=63° C.).

Vapam Treatment: LDPE films are placed in a close chamber and exposed to the vapors of a 0.74 v/v solution of VAPAM® (Sodium N-methyldithiocarbamate 39.1 WT %) in water. Final Volume 2.0 L. The system is kept at 30 C for 20 days. Then the LDPE films are subjected to light exposure as described above.

### Evaluation Parameters:

1) Carbonyl Increment: Evaluation of the carbonyl band increment (1710 cm−1) in LDPE films additivated at 0.4% with compound under applicative tests to assess the performances as light/heat stabilizers.
2) Tensile elongation break: Evaluation of elongation % property of LDPE films additivated at 0.4% with compound under applicative tests to assess the performances as light/heat stabilizers. Test carried out with ZWICK Z1.0 testing machine: speed: 100 mm/min; holder distance: 30mm; Temperature: 20° C.

TABLE B5
Carbonyl Increment of 150 μm additivated
LDPE films upon WOM exposure

| | Time of exposure (h) | | | | | | | |
|---|---|---|---|---|---|---|---|---|
| Formulation | 0 | 999 | 1993 | 3024 | 4009 | 4992 | 5994 | 7016 |
| Film 1 | 0.000 | 0 | 0.004 | 0.009 | 0.021 | 0.036 | 0.057 | 0.094 |
| Film 2 | 0.000 | 0 | 0.009 | 0.023 | 0.046 | 0.071 | 0.098 | 0.132 |
| Film 3 | 0.000 | 0 | 0.059 | 0.115 | 0.193 | 0.324 | 0.567 | — |

TABLE B6
Carbonyl Increment of 150 μm additivated LDPE films upon WOM-
Vapam exposure

Time of exposure (h)

| Formulation | 0 | 967 | 1542 | 2042 |
|---|---|---|---|---|
| Film 1 | 0 | 0.046 | 0.058 | 0.064 |
| Film 2 | 0 | 0.055 | 0.070 | 0.088 |
| Film 3 | 0 | 2.143 | — | — |

TABLE B7
Tensile measurements of 150 μm additivated LDPE films upon WOM
exposure

Time of exposure (h)

| Formulation | 0 | 1993 | 4009 | 5497 |
|---|---|---|---|---|
| Film 1 | 100 | 90 | 90 | 84 |
| Film 2 | 100 | 96 | 89 | 83 |
| Film 3 | 100 | 72 | 14 | — |

TABLE B8
Tensile measurements of 150 μm additivated LDPE films upon WOM-
Vapam exposure

| Formulation Table 4 | 0 | 967 | 2610 |
|---|---|---|---|
| Film 2 | 100 | 77 | 25 |
| Film 3 | 100 | 0 | — |

1. A compound of formula (I) 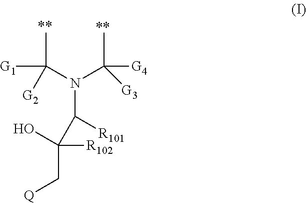
wherein:
G₁, G₂, G₃ and G₄ are independently alkyl groups having 1 to 4 carbon atoms, or G₁ and G₂, G₃ and G₄, or both, together are pentamethylene;
R₁₀₁ and R₁₀₂ are independently hydrogen or C₁-C₈alkyl;
Q is a group of formula (II), (III), (IV) or (V) 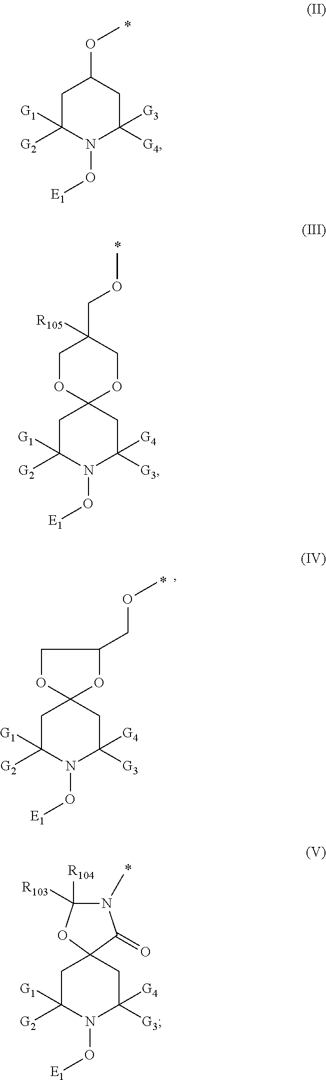
R₁₀₃ and R₁₀₄ are independently —C₃-C₇cycloalkyl or C₁-C₁₈alkyl, which is unsubstituted or substituted with halogen, nitro or —C(O)H;
R₁₀₅ is ethyl or methyl;
E₁ is straight or branched chain C₁-C₂₄alkyl, C₂-C₁₈alkenyl, C₂-C₁₈alkinyl, C₅-C₁₂cycloalkyl, C₅-C₁₂cycloalkenyl, phenyl, naphthyl or C₇-C₁₅phenylalkyl which are optionally unsubstituted or substituted with 1 to 3 OH groups, or
E₁ is C₂-C₂₄alkyl which is substituted by NO₂, POR′ or NHR′ wherein R′ is C₁-C₁₂alkyl;
* indicates a point of attachment; and
** indicates a point of attachment to one or two organic residues comprising 1-500 carbon atoms and optionally 1-200 heteroatoms.

2. The compound of claim 1 represented by formula (I′) 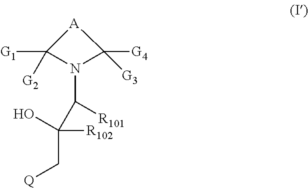
wherein A is a divalent group forming a cyclic or heterocyclic 5-, 6- or 7-membered ring, which is unsubstituted or substituted by —OH , ═O or by one or two organic residues comprising 1-500 carbon atoms and optionally 1-200 heteroatoms.

3. The compound of claim 1 represented by a compound selected from the group consisting of 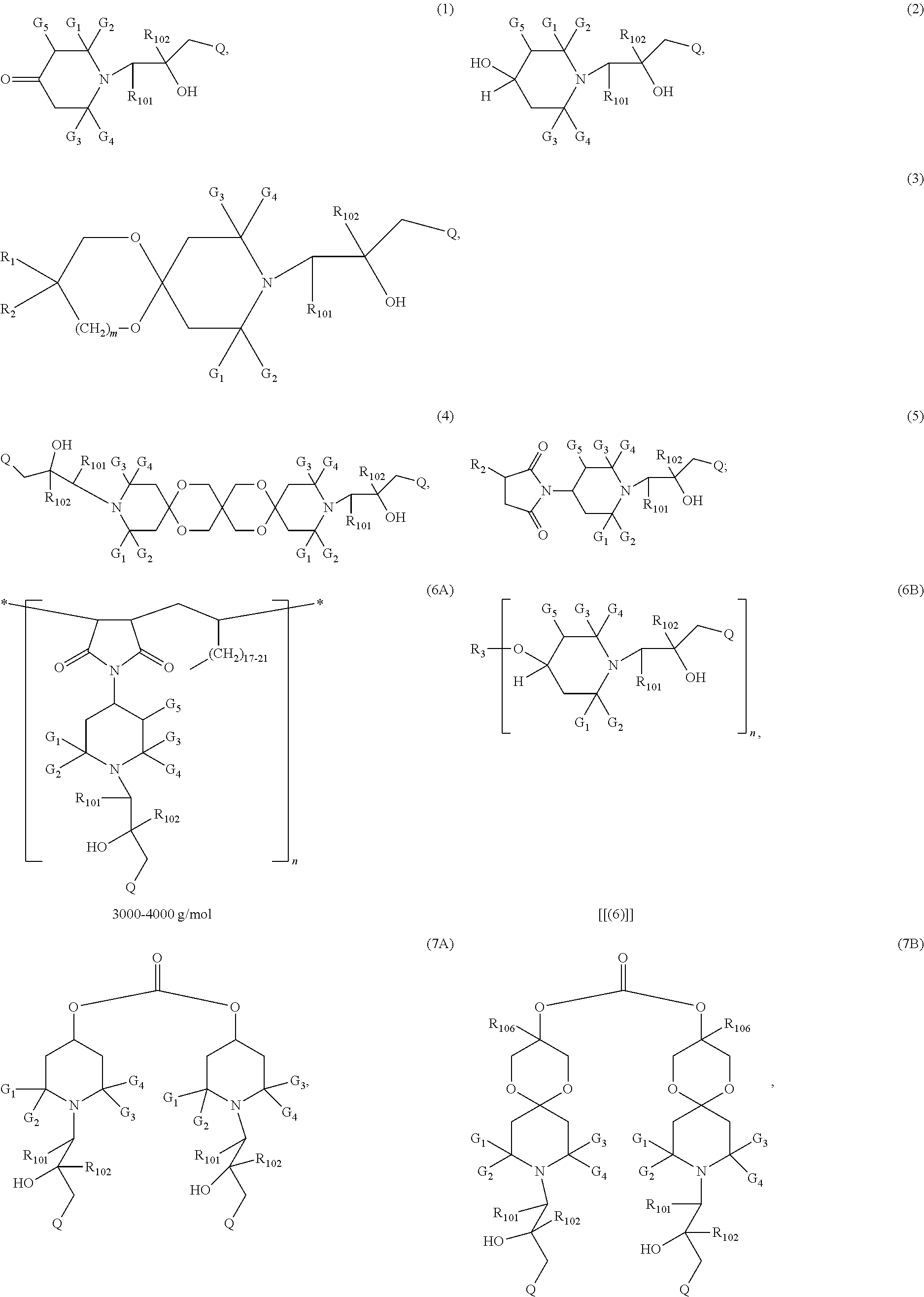 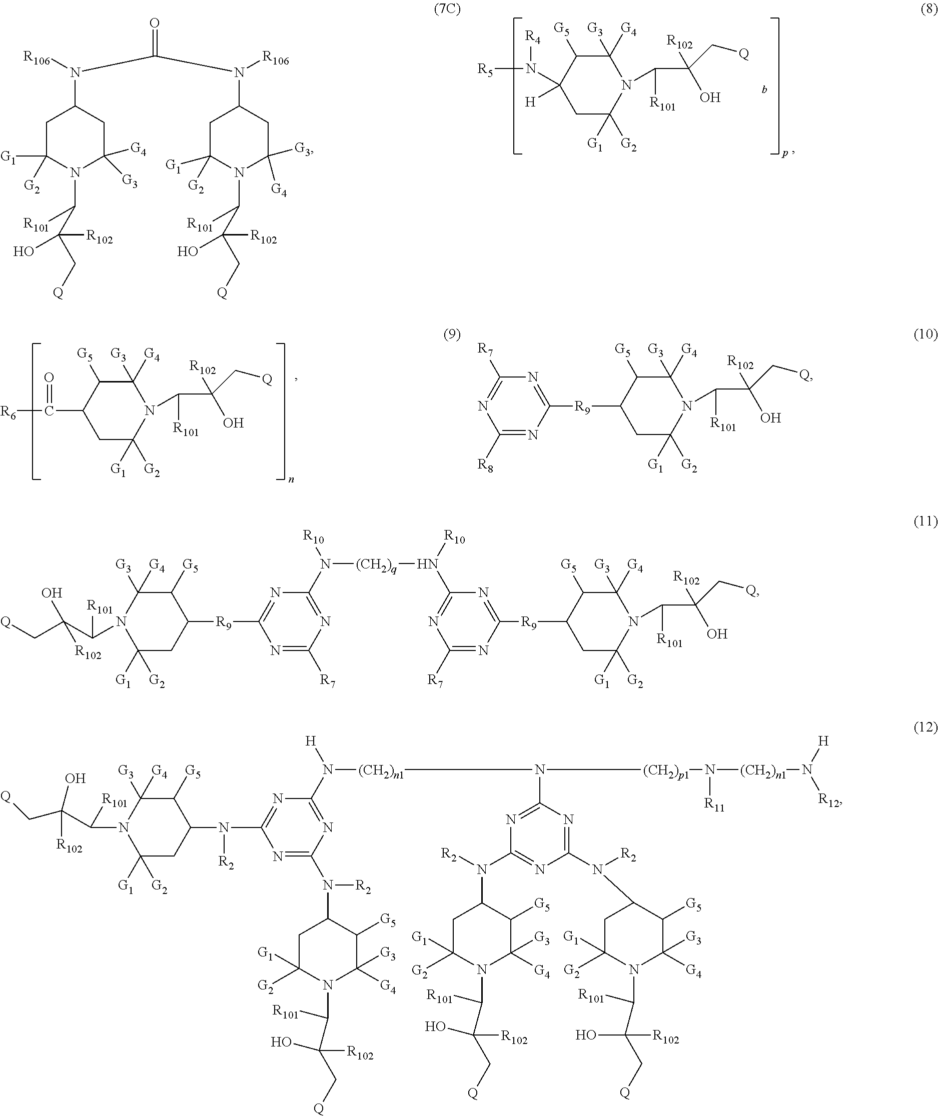 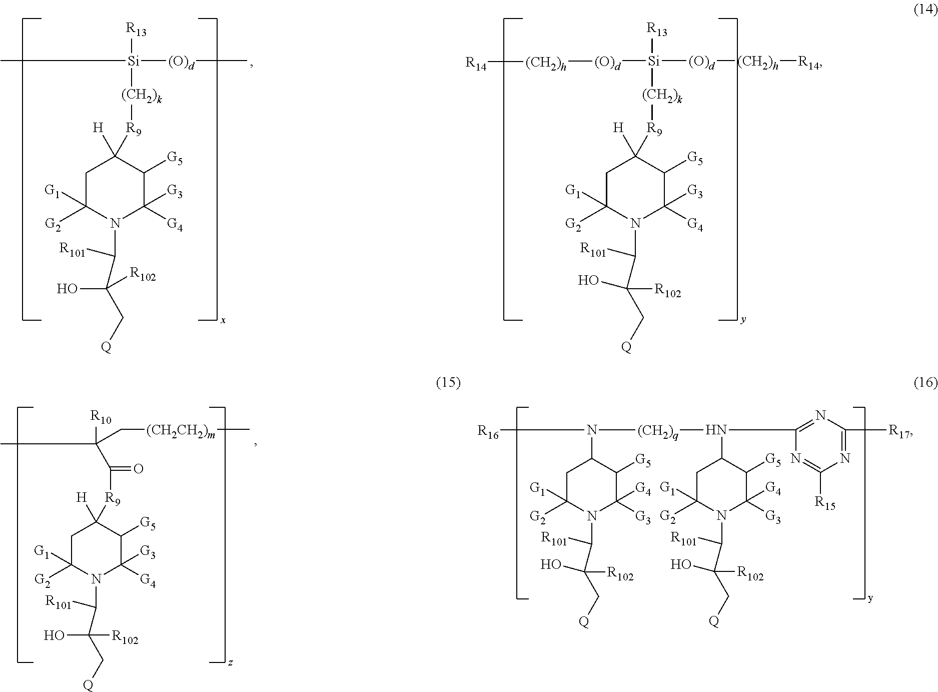
wherein:
G₁, G₂, G₃, G₄, R₁₀₁, R₁₀₂, R₁₀₃, R₁₀₄, R₁₀₅ and Q are defined as in claim 1;
G₅ is hydrogen or methyl;
R₁₀₆ is hydrogen, alkyl of 1 to 12 carbon atoms or alkenyl of 2 to 12 carbon atoms;
m is 0 or 1;
R₁ is hydrogen, hydroxyl or hydroxymethyl;
R₂ is hydrogen, alkyl of 1 to 12 carbon atoms or alkenyl of 2 to 12 carbon atoms;
n is 1 to 4;
when n is 1,
R₃ is alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid comprising 2 to 18 carbon atoms, an acyl radical of a cycloaliphatic carboxylic or carbamic acid comprising 7 to 12 carbon atoms, or acyl radical of an aromatic acid comprising 7 to 15 carbon atoms;
when n is 2,
R₃ is alkylene of 2 to 18 carbon atoms, a divalent acyl radical of an aliphatic or unsaturated aliphatic dicarboxylic or dicarbamic acid comprising 2 to 18 carbon atoms, a divalent acyl radical of a cycloaliphatic dicarboxylic or dicarbamic acid comprising 7 to 12 carbon atoms, or a divalent acyl radical of an aromatic dicarboxylic acid comprising 8 to 15 carbon atoms;
when n is 3,
R₃ is a trivalent acyl radical acyl radical of an aliphatic, unsaturated aliphatic, or cycloaliphatic tricarboxylic acid or tricarbamic acid comprising 6-18 carbon atoms, or a trivalent acyl radical of an aromatic tricarboxylic or tricarbamic acid comprising 9-18 carbon atoms, or R₃ is a trivalent acyl radical of a tris(alkylcarbamic acid) derivative of cyanuric acid comprising 12-24 carbon atoms;
when n is 4,
R₃ is a tetravalent acyl radical of an aliphatic or unsaturated aliphatic tetracarboxylic acid, or R₃ is a tetravalent acyl radical of an aromatic tetracarboxylic acid comprising 10 to 18 carbon atoms;
p is 1 to 3;
R₄ is hydrogen, alkyl of 1 to 18 carbon atoms or acyl of 2 to 6 carbon atoms;
when p is 1,
R₅ is hydrogen, alkyl of 1 to 18 carbon atoms, an acyl radical of an aliphatic or unsaturated aliphatic carboxylic or carbamic acid comprising 2 to 18 carbon atoms, an acyl radical of a cycloaliphatic carboxylic or carbamic acid comprising 7 to 12 carbon atoms, an acyl radical of an aromatic carboxylic acid comprising 7 to 15 carbon atoms, or R₄ and R₅ together are —(CH₂)₅CO—, phthaloyl or a divalent acyl radical of maleic acid;
when p is 2,
R₅ is alkylene of 2 to 12 carbon atoms, a divalent acyl radical of an aliphatic or unsaturated aliphatic dicarboxylic or dicarbamic acid comprising 2 to 18 carbon atoms, a divalent acyl radical of a cycloaliphatic dicarboxylic or dicarbamic acid comprising 7 to 12 carbon atoms, or a divalent acyl radical of an aromatic dicarboxylic acid comprising 8 to 15 carbon atoms;
when p is 3,
R₅ is a trivalent acyl radical of an aliphatic or unsaturated aliphatic tricarboxylic acid comprising 6 to 18 carbon atoms, or a trivalent acyl radical of an aromatic tricarboxylic acid comprising 9 to 15 carbon atoms;
when n is 1,
R₆ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms;
when n is 2,
R₆ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)— of 2 to 18 carbon atoms, or R₆ is 4-methyl-1,3-phenylenediamino;
when n is 3,
R₆ is a trivalent alkoxy radical of a saturated or unsaturated aliphatic triol comprising 3 to 18 carbon atoms;
when n is 4,
R₆ is a tetravalent alkoxy radical of a saturated or unsaturated aliphatic tetraol comprising 4 to 18 carbon atoms;
R₇ and R₈ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms;
R₉ is a divalent oxygen atom, or R₉ is a divalent nitrogen atom substituted by either hydrogen, alkyl of 1 to 12 carbon atoms or T₁ 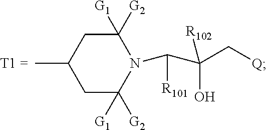
R₁₀ is hydrogen or methyl;
q is 2 to 8;
R₁₁ and R₁₂ are independently hydrogen or the group T₂ 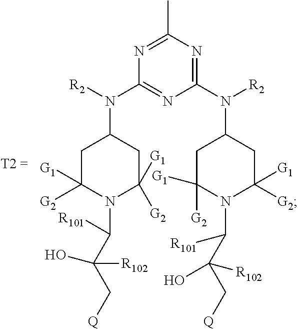
n1 is a number from 1 to 6;
p1 is a number from 1 to 6;
R₁₃ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₄ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
y is 1 to 10;
z is an integer such that the compound has a molecular weight of 1000 to 4000 amu;
R₁₅ is morpholino, piperidino, 1-piperizinyl, alkylamino of 1 to 8 carbon atoms, —N(alkyl)T₁ with alkyl of 1 to 8 carbon atoms substituted by T₁ or —N(alkyl)₂ of 2 to 16 carbon atoms;
R₁₆ is hydrogen, acyl of 2 to 4 carbon atoms, carbamoyl substituted by alkyl of 1 to 4 carbon atoms, s-triazinyl substituted once by chlorine and once by R₁₅, or s-triazinyl substituted twice by R₁₅ with the condition that the two R₁₅ substituents are optionally different;
R₁₇ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃ 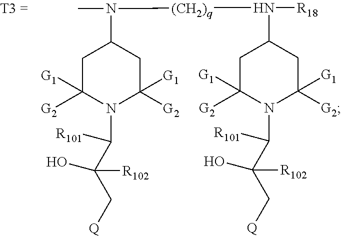
q is 2 to 8; and
R₁₈ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms.

4. The compound of claim 1, wherein
G₁ and G₃ are ethyl and G₂, G₄ and G₅ are methyl, or
G₁ and G₂ are methyl, G₃ and G₄ are ethyl and G₅ is hydrogen or G₁, G₂, G₃ and G₄ are methyl and G₅ is hydrogen.

5. The compound of claim 1, wherein
G₁, G₂, G₃ and G₄ are methyl,
R₁₀₁ and R₁₀₂ are hydrogen,
and R₁₀₃ and R₁₀₄ are independently C₁-C₁₈alkyl.

6. The compound claim 1, wherein Q is a group of formula (II) or (III).

7. The compound of claim 1, wherein E₁ is straight or branched C₁-C₁₈alkyl.

8. The compound of claim 3 which is of formulae (1), (2), (3), (6A), (6B), (7A), (7B), (10) (12) or (16).

9. A process for preparing the compound of formula (I) 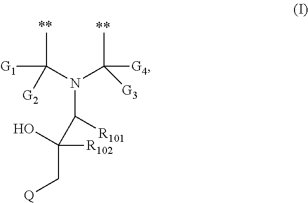
the process comprising reacting a sterically hindered amine comprising the structural element of formula (VI) 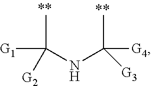
wherein:
G₁, G₂, G₃ and G₄ are independently alkyl of 1 to 4 carbon atoms, or G₁ and G₂, G₃ and G₄, or both, together are pentamethylene;
** indicates a point of attachment to one or two organic residues comprising 1-500 carbon atoms and optionally 1-200 heteroatoms, with a compound of formula (VII) 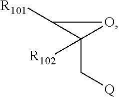
wherein:
R₁₀₁ and R₁₀₂ are independently hydrogen or C₁-C₈alkyl;
Q is a group of formula (II), (III), (IV) or (V) 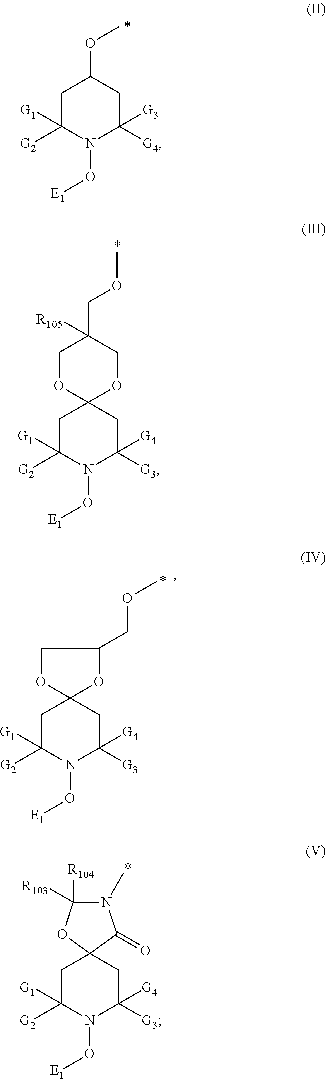
R₁₀₃ and R₁₀₄ are independently —C₃-C₇cycloalkyl or C₁-C₁₈alkyl, which is unsubstituted or substituted with halogen, nitro or —C(O)H;
R₁₀₅ is ethyl or methyl;
E₁ is straight or branched chain C₁-C₂₄alkyl, C₂-C₁₈alkenyl, C₂-C₁₈alkinyl, C₅-C₁₂cycloalkyl, C₅-C₁₂cycloalkenyl, phenyl, naphthyl or C₇-C₁₅phenylalkyl which are optionally unsubstituted or substituted by 1 to 3 OH groups; and
* indicates the point of attachment,
in the presence of a Lewis acid as catalyst.

10. The process of claim 9 carried out at a temperature from 20° C. to 280° C.

11. A composition, comprising:
(a) an organic polymer subject to adverse effects of heat, oxygen and light; and
(b) at least one compound of formula (I) of claim 1.

12. The composition of claim 11, wherein the polymer (a) is a thermoplastic organic polymer or a coating binder.

13. The composition of claim 11, further comprising at least one additional component selected from the group consisting of a solvent, a pigment, a dye, a plasticizer, an antioxidant, a thixotropic agent, a levelling assistant, a further light stabilizer, a metal passivator, a metal oxide, an organophosphorus compound, a hydroxylamine, a UV absorber, and a sterically hindered amine.

14. A process for stabilizing an organic polymeric material against damage by light, oxygen and/or heat, the process comprising adding to, or applying to, said material at least one compound of formula (I) of claim 1.

15. A flame retardant, comprising the compound of claim 1.

16. The compound of claim 3, wherein
when n is 3,
R₃ is 1,3,5-tris[6-carboxyaminohexyl]-2,4,6-trioxo-s-triazine.

17. The compound of claim 3, wherein
R₁₅ is a branched alkylamino of 3 to 8 carbon atoms.

18. A composition, comprising:
(a) an organic polymer subject to adverse effects of heat, oxygen and light; and
(b) at least one compound of formula (I′) of claim 2.

19. A composition, comprising:
(a) an organic polymer subject to adverse effects of heat, oxygen and light; and
(b) at least one compound of formulae 1 to 16 of claim 3.

20. A process for stabilizing an organic polymeric material against damage by light, oxygen and/or heat, the process comprising adding to, or applying to, said material at least one compound of formula (I′) of claim 2.
